(19) United States
(12) Patent Application Publication
(10) Pub. No.: US 2013/0071329 A1
(43) Pub. Date: Mar. 21, 2013

(54) THERANOSTIC DELIVERY SYSTEMS WITH MODIFIED SURFACES

(71) Applicants: Mauro Ferrari, Houston, TX (US); Ennio Tasciotti, Houston, TX (US); Nicoletta Quattrocchi, Houston, TX (US)

(72) Inventors: Mauro Ferrari, Houston, TX (US); Ennio Tasciotti, Houston, TX (US); Nicoletta Quattrocchi, Houston, TX (US)

(73) Assignee: Board of Regents of the University of Texas System, Austin, TX (US)

(21) Appl. No.: 13/635,530

(22) PCT Filed: Mar. 17, 2011
(86) PCT No.: PCT/US11/28861

§ 371 Date: Nov. 30, 2012

Related U.S. Application Data
(60) Provisional application No. 61/282,688, filed on Mar. 17, 2010.
(60) Provisional application No. 61/282,691, filed on Mar. 17, 2010.

(51) Int. Cl. A61K 9/14; A61K 49/00; A61K 9/127

(52) U.S. Cl. CPC A61K 9/14; A61K 9/1271; A61K 49/00
USPC 424/9.1; 424/490; 424/450; 424/493; 427/2.14

(57) ABSTRACT

The present invention pertains to therapeutic compositions and delivery systems comprising at least one microparticle or nanoparticle. In various embodiments, the surface of the microparticle or nanoparticle is modified or functionalized with at least a portion of an isolated cellular membrane, such as an isolated plasma membrane. In addition, the microparticle or nanoparticle contains at least one active agent, such as a therapeutic and/or imaging agent. In additional embodiments, the compositions and delivery systems of the present invention may be used for targeted delivery of an active agent. Also provided are methods of making the therapeutic compositions and delivery systems of the present invention.

### CROSS-REFERENCE TO RELATED APPLICATIONS

This application claims priority to U.S. Provisional Patent Application Nos. 61/282,688 and 61/282,691, both filed on Mar. 17, 2010. This application is also related to the PCT Application entitled “Universal Cell-directed Theranostics”, filed concurrently herewith on Mar. 17, 2011. The entirety of each of the above-identified applications are incorporated herein by reference.

### STATEMENT REGARDING FEDERALLY SPONSORED RESEARCH

This invention was made with government support under Grant No. NNJ06HE06A, awarded by the National Aeronautics and Space Administration; and Grant Nos. W81XWH-09-2-0139 and W81XWH-07-2-0101, both awarded by the U.S. Department of Defense. The government has certain rights in the invention.

### BACKGROUND OF THE INVENTION

Systems and compositions for delivering active agents to desired sites in organisms have numerous therapeutic, preventive, imaging, and diagnostic applications. Current systems and compositions for achieving such tasks suffer from numerous limitations, including specificity and efficacy. Therefore, there is currently a need to develop more effective systems and compositions for delivering active agents to desired sites in organisms.

### BRIEF SUMMARY OF THE INVENTION

In some embodiments, the present invention provides compositions and delivery systems that comprise at least one microparticle or nanoparticle. The microparticle or nanoparticle further comprises at least: (1) one active agent (e.g., therapeutic agent or imaging agent); and (2) a surface. The surface also comprises at least a portion of an isolated cellular membrane, such as a plasma membrane. In various embodiments, the microparticle or nanoparticle is a lipid particle or a liposome that contains a lipid layer. In various embodiments, the lipid layer of the lipid particle or liposome also comprises a portion of the isolated cellular membrane. In additional embodiments, the microparticle or nanoparticle is a fabricated particle, a porous particle (e.g., a porous silicon or a porous silica) or a multistage object.

Additional embodiments of the present invention pertain to methods of making the aforementioned compositions as delivery agents. Such methods generally comprise: (1) isolating a cellular membrane from a cell; and (2) associating at least a portion of the isolated cellular membrane with a surface of a microparticle or a nanoparticle. Additional embodiments of the present invention pertain to delivery methods that comprise administering to a subject the compositions and delivery agents of the present invention.

### BRIEF DESCRIPTION OF THE FIGURES

In order that the manner in which the above recited and other advantages and objects of the invention are obtained, a more particular description of the invention briefly described above will be rendered by reference to specific embodiments thereof, which are illustrated in the appended Figures. Understanding that these Figures depict only typical embodiments of the invention and are therefore not to be considered limiting of its scope, the invention will be described with additional specificity and detail through the use of the accompanying Figures in which:

FIGS. 1A-1F schematically illustrate methods of making and using delivery systems in accordance with various embodiments of the present invention. Specifically, the delivery systems in this example include a microparticle or nanoparticle with a surface that is modified or functionalized with a cellular membrane of an immune cell. Such delivery system may be referred to as a “Leukolike” system.

FIG. 1A illustrates obtaining a blood sample from a donor animal, which may be a mammal. The blood sample contains immune cells, which may be first isolated and then ex vivo expanded, genetically modified and used for the surface modification or functionalization of the delivery system. Different cell types from the blood (peripheral blood mononuclear cells) can be isolated through cytofluorimetry sorting, magnetic beads and other affinity assays in order to derive specific membranes.

FIG. 1B illustrates isolation of plasma membranes from the isolated immune cells from the blood sample obtained in FIG. 1A. The isolation of plasma membrane may be performed by achieved through ultracentrifugation across a discontinuous sucrose density gradient. The plasma membrane may be identified using one or more markers specific to a plasma membrane. Such markers may be CD45, CD3z, LFA1 and CD20R. As also shown, one may use a dot blot technique for the plasma membrane identification.

FIG. 1C schematically shows a delivery system whose surface is modified or functionalized with a plasma membrane isolated from the immune cells shown in FIGS. 1A-1B. The delivery system in FIG. 1C contains a load (schematically shown as internal dots), which may be an active agent, such as a therapeutic agent and/or an imaging agent. In some embodiments, the load may be a second stage particle, which may contain an active agent. As shown in FIG. 1C, the modified or functionalized delivery system in this specific and non-limiting embodiment expresses on its surface all the proteins that were expressed on the immune cell used for the surface modification or functionalization.

FIG. 1D schematically shows the modified or functionalized delivery system in a vasculature of a recipient subject, to whom the particle has been administered. The recipient subject may or may not be the same subject as the donor of the blood sample. FIG. 1D schematically shows that the modified or functionalized delivery system may be able to avoid an uptake by macrophages of the reticuloendothelial system similarly to natural leukocytes in the blood system.

FIG. 1E schematically shows the modified or functionalized delivery system recognizing a target site and penetrating the endothelial cells of the recipient subject's vasculature. In this example shown, the target site is a tumor site, as indicated by tumor specific protein(s) on its surface

FIG. 1F schematically shows the modified or functionalized delivery system releasing its load at the tumor site.

FIG. 2 illustrates leukocyte plasma membrane isolation (left) by ultracentrifugation through a discontinuous sucrose density gradient followed by protein characterization (right) using a dot blot technique. Dots in the boxes represent fractions containing the leukocytes' cellular membranes that were used for the modification of functionalization of porous silicon particles.

FIG. 3 shows TEM images of leukocyte plasma membranes, nanoporus silicon particles (NSPs) and assembled leukolike systems.

FIG. 3A shows leukocyte plasma membranes isolated by ultracentrifugation through discontinuous sucrose density gradient. The membranes spontaneously organize into lipid vesicles, with a diameter size ranging from 200 nm to 1 μm. The lipid vesicles can be constituted by one or more lipid bilayers.

FIG. 3B shows how NSPs look before the coating with the leukocyte membranes.

FIG. 3C shows leukolike systems constituted by NSPs coated with isolated leukocyte membranes.

FIG. 3D shows a close up of the leukolike systems showing the interaction between membrane lipid vesicles and the NSPs surface. The lipid vesicles are constituted by more than one lipid bilayer that are not still spread onto the NSPs surface.

FIG. 3E shows a top view of a leukolike system.

FIG. 4 shows scanning electron microscopy (SEM) images of NSPs and leukolike systems. Top and bottom sides of the images show the nanoporus surface. Micrographs of different leukolike systems show different membrane coating efficiencies. The membrane coating efficiency is correlated to the concentration ratio of membrane:NSPs used during the coating step.

FIG. 4A shows an SEM image of an uncoated NSP (front face).

FIG. 4B shows an SEM micrograph of a leukolike system with a surface not completely coated by the isolated plasma membranes.

FIG. 4C shows a more focused SEM image of the leukolike system in FIG. 4B.

FIG. 4D shows an SEM image of another uncoated NSP (back face).

FIG. 4E shows an SEM image of a leukolike system with a surface completely coated by isolated plasma membranes.

FIG. 4F shows a more focused SEM image of the leukolike system showed in FIG. 4E.

FIG. 5 shows results of fluorescent activated cell sorting (FACS) of various cells.

FIG. 6 shows kinetics of canine T-cell expansion on K562-aAPC cells. Insert shows, by flow cytometry, that expanded cells are mixtures of CD4⁺ and CD8⁺ T-cells.

FIG. 7 shows FACS analyses of OKT-3/IL-2 activated T-cells (upper bracket) and JurkaT-cells that were electroporated with CD19-specific CAR mRNA (synthesized from T7 based DNA plasmid vectors). T-cells were analyzed with 2D3 Alexa-labeled CAR-specific mAb and T-cells marker CD8 after 24 hours of electroporation. Propidium iodide (PI) staining was used to determine the viability of the cells after electroporation.

FIG. 8 shows another leukocyte plasma membrane isolation scheme.

FIG. 8A shows membrane isolation through a discontinuous sucrose density gradient and immunoblotting of specific cellular membrane markers along the gradient fractions. In the white boxes are indicated the fractions containing the plasma cellular membranes enriched in the interested proteins LFA1 and CD3z. The cellular lysate was used As positive control, the 55% sucrose solution as negative control.

FIG. 8B shows TEM of leukocyte isolated membranes organized into lipid vesicles.

FIG. 8C shows particulars of FIG. 8B showing the lipid bilayer structure of a singular vesicle.

FIG. 9 shows the characterization of isolated leukocyte membranes.

FIGS. 9A-9E show TEM and SEM micrographs showing the adsorption of isolated leukocyte membranes on NSPs. The coating efficiency depends on the lipid concentration 1:5 (C), 1:2 (D), 1 (E) of the membrane solutions. In TEM are shown both the coronal and transversal sections of the bare NSPs (B) and LS (C-E). The SEM micrographs show how the porous surface of NSPs looks before and after coating with membrane solutions containing a different lipid concentration. The different coating efficiencies are clearly visible in the corresponding magnifications, on the right.

FIG. 9F shows that the size distribution of the NSPs diameter does not change after membrane coating as shown in the graph.

FIG. 10 shows additional characterizations of the isolated leukocyte membranes.

FIG. 10A shows data relating to net surface charge (zeta potential) reading for the isolated membranes, NSPs before and after APTES surface functionalization, LS and Jurkat cells.

FIG. 10B shows SEM micrographs of the LS (b and d) realized using oxidized- (a) or APTES-modified NSPs (c). The images show how the different surface net charge of the NSPs surface plays an important role in the interaction with the isolated membranes.

FIG. 10C show 3D reconstitution of NSPs (a) and SEM micrographs of LS made with different coating procedures (sonication (b), no-sonication (c)) and of a real leukocyte.

FIGS. 10D-10E show Flow cytometry analysis and immunoblotting showing the protein (CD3z, LFA1) composition of the LS surface (histograms on the right) in comparison with the Jurkat cells (histograms on the left).

FIG. 11 summarizes various experimental results relating to NSP and LS uptake in various cells.

FIGS. 11A-11B show flow cytometry analysis and corresponding histograms of macrophage-LS (green) and Jurkat-LS (red) uptake rate in the presence of J774A.1.

FIG. 11C shows confocal microscopy of macrophage-LS (green) and Jurkat-LS (red) (upper row), and NSPs (lower row) uptake rate in presence of J774A.1 after 3, 6 and 24 hr of incubation. In the lower row NSPs were labeled by loading bovine serum albumin conjugated to fluorescein isothiocyanate (FITC-BSA) (green)

FIG. 11D shows SEM micrographs of Jurkat-LS (upper row) and macrophage-LS (lower row) uptake rate in presence of J774A.1 at 3, 6 and 24 hr respectively.

FIG. 11E summarizes results relating to pro-inflammatory cytokines (TNF-α, IL-6) production by murine macrophages treated with zymosan suspension of 1 ng/ml and macrophage-LS for 3, 6 and 24 hr. TNF-α and IL-6 levels were assayed by ELISA. Data are representative of 3 experiments.

FIG. 12 shows the interaction of NSPs and LSs with lysosomes.

FIGS. 12A-12B show TEM micrographs and confocal images showing NSPs colocalization with lysosomes (left column, FIG. 12A) and LS localization into the cytoplasm (right column, FIG. 12B) after internalization by HUVECs at 2 h (upper panels), 4 h (middle panels), 24 h (lower panels). In the confocal images lysosomes were stained with Lysotracker Red (1 uM) for 1 h, NSPs are shown trough bright field while the LS is labeled with green fluorescent lipids. A magnification of each boxed region is shown at the corner of the correspondent panel.

FIG. 13 summarizes studies related to the release profiles of NSPs and LSs.

FIGS. 13A-13B show DOX- and BSA-release profile from NSPs (red) and LS (green). The release profiles have been checked in PBS pH 7.4 in moving condition for few days. A burst release at 0.5, 1 and 1.5 hr is shown in the inserts. All the experiments were done in triplicate.

FIG. 13C shows confocal microscopy images of LS loaded with FITC-BSA (a) and coated with leukocyte membranes stained with a rhodamine-lipid (b). The correspondent merge and bright field are shown in the panels c and d.

FIG. 13D shows confocal microscopy images of FITC-BSA (green) release from NSPs and LS (described in A) after 2, 24 and 48 hr of internalization with HUVECs. The FITC-BSA release starts at 24 hr prevalently from NSPs and it is more evident after 48 hr, as seen in the upper panels showing only the channel of the FITC-BSA. Some FITC-BSA from LS can be poorly observed after 48 hr. At 24 hr the coating membranes start to dissociate from the LS as shown by the spreading of the red fluorescence in the lower panels.

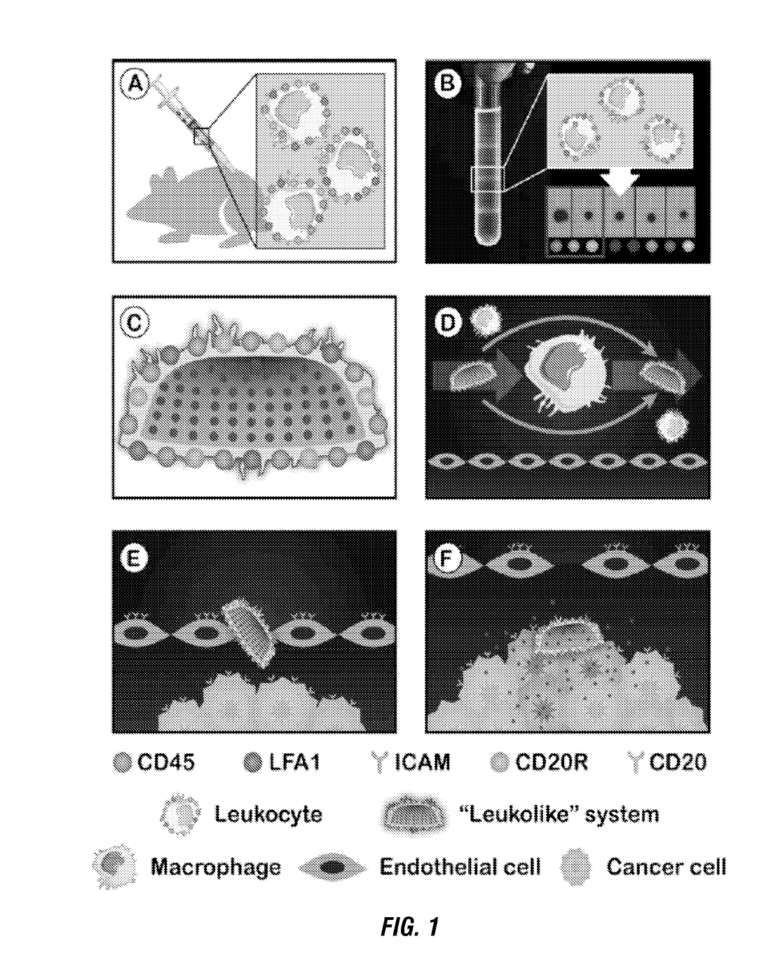

### DETAILED DESCRIPTION OF EXEMPLARY EMBODIMENTS

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

### RELATED APPLICATIONS AND PUBLICATIONS

The following research articles and patent documents, which are all incorporated herein by reference in their entirety, may be useful for understanding the present invention: (1) PCT Publication No. WO 2007/120248, published on Oct. 25, 2007; (2) PCT Publication No. WO 2008/041970, published on Apr. 10, 2008; (3) PCT Publication No. WO 2008/021908, published on Feb. 21, 2008; (4) U.S. Patent Application Publication No. 2008/0102030, published on May 1, 2008; (5) U.S. Patent Application Publication No. 2003/0114366, published on Jun. 19, 2003; (6) U.S. Patent Application Publication No. 2008/0206344, published on Aug. 28, 2008; (7) U.S. Patent Application Publication No. 2008/0280140, published on Nov. 13, 2008; (8) PCT Patent Application PCT/US2008/014001, filed on Dec. 23, 2008; (9) U.S. Pat. No. 6,107,102, issued on Aug. 22, 2000; (10) U.S. Patent Application Publication No. 2008/0311182, published on Dec. 18, 2008; (11) PCT Patent Application PCT/US2009/000239, filed on Jan. 15, 2009; (12) PCT Patent Application PCT/US2011/27746, filed on Mar. 9, 2011; (13) U.S. Patent Application Publication No. 2010/0029785, published on Feb. 4, 2010; (14) Tasciotti E. et al. 2008. Nature Nanotechnology. 3:151-157; and (15) PCT Application entitled “Universal Cell-Directed Theranostics”, concurrently being filed herewith.

### DEFINITIONS

Unless otherwise specified, “a” or “an” means one or more.

“Microparticle” means a particle having a maximum characteristic size from 1 micron to 1000 microns, or from 1 micron to 100 microns.

“Nanoparticle” means a particle having a maximum characteristic size of less than 1 micron.

“Nanoporous” or “nanopores” refers to pores with an average size of less than 1 micron.

“Biodegradable material” refers to a material that can dissolve or degrade in a physiological medium, such as PBS or serum.

“Biocompatible” refers to a material that, when exposed to living cells, will support an appropriate cellular activity of the cells without causing an undesirable effect in the cells, such as a change in a living cycle of the cells; a release of pro-inflammatory factors; a change in a proliferation rate of the cells; and a cytotoxic effect.

APTES stands for 3-aminopropyltriethoxysilane.

PEG refers to polyethylene glycol.

FBS stands for fetal bovine serum.

SEM stands for scanning electron microscope.

TEM stands for transmission electron microscope.

Physiological conditions stand for conditions, such as the temperature, osmolarity, and pH close to that of plasma conditions of a healthy mammal, such as a healthy human being.

The term “theranostic” refers to a delivery system, which may be used to at least one of treating, preventing, monitoring or diagnosing of a physiological condition or a disease.

The terms “isolated cellular membrane” and “cellular membrane” refer to either complete or incomplete portions of cellular membranes that may or may not be in native form, shape, composition and/or organization.

Introduction

In some embodiments, the present invention provides compositions and delivery systems that comprise at least one microparticle or nanoparticle. The microparticles or nanoparticles further comprise at least: (1) one active agent (e.g., therapeutic agent or imaging agent); and (2) a surface. The surface also comprises at least a portion of an isolated cellular membrane.

Additional embodiments of the present invention pertain to methods of making the aforementioned compositions as delivery systems. Such methods generally comprise: (1) isolating a membrane from a cell; and (2) associating at least a portion of the isolated cellular membrane with a surface of a microparticle or a nanoparticle. Additional embodiments of the present invention pertain to delivery methods that comprise administering to a subject the compositions and delivery systems of the present invention. Various aspects of the aforementioned embodiments will now be described in more detail as specific and non-limiting examples.

Isolated Cellular Membranes and their Therapeutic/Diagnostic Effects

In many embodiments, the isolated cellular membranes of the present invention may be isolated plasma membranes, isolated nuclear membranes, or isolated mitochondrial membranes. In many embodiments, it may be preferred to use an isolated plasma membrane for surface modification or functionalization.

The isolated cellular membranes of the present invention may constitute complete or incomplete portions of cellular membranes. Furthermore, the isolated cellular membranes of the present invention may or may not be in native form, shape, composition and/or organization. In some embodiments, the isolated cellular membranes of the present invention may constitute at least a portion of a native or isolated cellular membrane in terms of form, shape, composition and/or organization.

In various embodiments, the isolated cellular membranes may be derived from the cells of living organisms, such as animals or plants. In many embodiments, the isolated cellular membranes may be derived from cells of a warm blooded animal, such as a bird or a mammal. In certain embodiments, the isolated cellular membranes may be derived from human cells.

In some embodiments, the isolated cellular membranes may be derived from mammalian cells, such as human cells. In more specific embodiments, the isolated cellular membranes may be derived from immune cells, such as genetically modified immune cells. In further embodiments, the isolated cellular membranes may be derived from T-cells, Natural killer (NK) cells, monocytes, leukocytes and macrophages.

In additional embodiments, the isolated cellular membranes may be derived from immune cells such as neutrophils; eosinophils; basophils; lymphocytes, such as a B-cells, T-cells or NK cells; monocytes; macrophages; or dendritic cells. In some embodiments, the isolated cellular membrane may be derived from a T-cell, such as a monoclonal T-cell or a polyclonal T-cell. In some embodiments, the T-cell may be tumor-antigen specific T-lymphocyte. In some embodiments, the cell may be a cytotoxic T-cell, such as an activated cytotoxic T-cell. In some embodiments, the isolated cellular membrane may be derived from cytotoxic lymphocytes, NK cells, monocytes, and/or macrophages.

In some embodiments, the cell from which the cellular membrane is derived from (such as a T-cell, NK cell, monocyte or macrophage) may be isolated from a blood of a donor subject. In some embodiments, the donor subject may be an animal, such as a warm blooded animal (e.g., a bird, or a mammal, such as a human). As discussed in more detail below, delivery systems with surfaces that are modified with at a least portion of cellular membranes from such immune cells are useful for delivering active agents (e.g., a therapeutic agent and/or an imaging agent) to a subject.

In some embodiments, the subject donating the cellular membrane may be a donor subject with a condition or a disease. In some embodiments, such conditions or diseases may be associated with an inflammation-related disease. In other embodiments, such conditions may be cancerous conditions. Thus, in such embodiments, delivery systems containing isolated cellular membranes from such subjects can be used to treat or monitor the condition or disease that the donor suffered from when the delivery system is administered to a recipient subject. In particular, and without being bound by theory, such monitoring or treatment can occur because the delivery systems may be able to target a body site in the recipient subject that is associated with the condition or disease that the donor subject was suffering from.

In some embodiments, the immune cell from which the cellular membrane is derived may be a genetically modified immune cell. For example, the genetically modified immune cell may be a cell modified as detailed in the section “Cell modification” below.

In additional embodiments, the isolated cellular membranes of delivery systems may be derived from an immune cell. In such a case, the target-oriented properties of the immune cell may be transferred on the functionalized or modified delivery system.

In some embodiments, an ability of the functionalized or modified delivery system to target a diseased site may be improved by using a retargeting strategy. In some embodiments, this may involve genetic modification of an immune cell from the diseased site, and the isolation of the cellular membrane from the genetically modified immune cell for surface functionalization or modification of the delivery system. For example, for targeting a tumor or metastasis, genetically transformed cells that are related to a T-cell gene receptor (TCR) or other tumor-specific antibodies may be used as sources for cellular membranes.

In operation, delivery systems functionalized or modified with the above-described cellular membranes may head towards a diseased site, such as tumor site. The delivery system may then exert a therapeutic, imaging or diagnostic effect. This may occur by the release of an active agent (e.g., a therapeutic and/or imaging agent) from the delivery system. Such release may be triggered by a specific internal or external factor. Such factors may include physical or physiological factors. Examples of changes that may trigger an active agent release may include changes in pH, pressure, or temperature. Furthermore, such release may occur in a time-dependent manner, such as by degradation of the outer surface of the particles by cytoplasmic enzymes, lysosomes, endosomes, or by changes in pH.

In additional embodiments, the cells from which cellular membranes are derived from are naturally occurring cells, such as differentiated cells. For example, the cell may be a naturally occurring differentiated cell from a warm blooded animal, such as a bird, a mammal, or a human. In more specific embodiments, the cell may be a naturally occurring, differentiated cell from a human body. Yet, in some embodiments, the cells from which cellular membranes may be derived from may be naturally occurring but non-differentiated cells, such as stem cells.

In additional embodiments, one may use a selection of cells to isolate cellular membranes. The isolated cellular membranes may then be used to modify or functionalize a delivery system. In some embodiments, the delivery system may then be introduced into a mammal's body for the controlled release of an active agent to a disease site.

In some embodiments, the active agent may be a cytostatic drug and/or an angiogenesis inhibitor; an antibody-based therapeutic agent; therapeutic DNA for transfection; an RNAi-based therapeutic agent, which is selectively aimed to genes and viruses causing a disease; and chemokines, such as cytokines. In some embodiments, the active agent may be an imaging agent, such as ferromagnetic particles (NMR-diagnostics); quantum dots; or metal nanoparticles, such gold nanoparticles. Additional active agents, which may be used are disclosed in the section entitled “Active Agents” below.

In some embodiments, the modified or functionalized delivery system may be used for both therapeutic and imaging or diagnostic purposes. This may be accomplished, for example, by combining a therapeutic agent and an imaging agent in the modified delivery system. This may also be accomplished by administering a modified delivery system that includes a first faction loaded with a therapeutic agent and a second faction loaded with an imaging agent.

Modification of Cells Prior to Cellular Membrane Isolation

In additional embodiments, prior to isolating a cellular membrane, the isolated cell(s) may be modified. In some embodiments, the modification involves ex vivo expansion of cells. In some embodiments, the cells are immune cells. In such embodiments, cellular membranes may be isolated from immune cells obtained by such ex vivo expansion.

In additional embodiments, prior to isolating a cellular membrane, the isolated cell(s) may be modified in order to enhance their targeting capability towards a target site, such as a site affected by a disease. In such embodiments, the modified cell(s) can have a greater ability to recognize and/or find cells of the target site, such as, for example, tumor cells. As discussed below, the enhancement of the targeting capability of the isolated cell may be realized in a number of different ways.

For instance, in some embodiments, an isolated cell (e.g., an immune cell) may be expanded ex vivo and genetically modified in such a way that its membrane (e.g., plasma membrane) expresses a specific receptor for a protein that is expressed (or over-expressed) at a target site (e.g., an inflamed site or a tumor site). In some embodiments, the genetic modification may involve introducing a gene of interest into the genome of the expanded cell(s) by genetic transfer. Such genes of interest may be derived from a cell in a desired target site, such as a tumor site. For example, to enhance tumor targeting ability, one can introduce in the genome of the expanded cell(s) a T-cell receptor gene (TCR gene) from a tumor associated antigen (TAA) specific immune cell, such as a TAA specific T-cell.

In more specific embodiments, immune cells, such as T-cells, may be genetically modified to redirect specificity to desired tumor-associated antigens (TAA) using a chimeric antigen receptor (CAR) that recognizes TAA independently of the major histocompatibility complex. This may be accomplished by using one of the following two technologies: (i) Sleeping Beauty (SB) transposon/transposase to stably express a CAR from DNA; and (ii) artificial antigen presenting cells (aAPC) adapted from K562 cells to efficiently and selectively propagate CAR⁺ T-cells ex vivo.

By way of background, SB is a gene-insertion system that is capable of mediating the transposition of DNA sequences from transfected plasmids into vertebral cell chromosomes. It may include a transposon, composed of the gene of interest, and a hyperactive transposase. The SB system may be used to improve non-viral gene transfer [14-17, see REFERENCES LIST 2 below] The SB system received regulatory approval for the in-human use of the SB system to genetically modify T-cells [18].

In some embodiments, immune cells, such as T-cells, may be genetically modified using the SB system to express a CAR against B-lineage lymphoma TAAs CD19 and CD20. For example, one may use the SB transposon/transposase system to improve DNA plasmid integration efficiency after T-cell electroporation.

In some embodiments, the enhancement of the targeting capability of the isolated immune cell(s) may be performed by combining the isolated immune cell(s) with one or more antibodies, which are specific to the target site. Such combining may involve incorporating the one or more antibodies on the lipid layer of the plasma membrane of the isolated cell. In some embodiments, when the target site comprises coopted vasculature, the isolated immune cell(s) may be combined with an antibody to angiopoietin 2. Likewise, when the target site comprises angiogenic vasculature, the isolated immune cell(s) may be combined with an antibody to vascular endothelial growth factor (VEGF); an antibody to fibroblast growth factor (FGFb); or an antibody to an endothelial marker, such as αᵥβ₃ integrins.

In other embodiments, when the target site comprises a renormalized vasculature, the isolated immune cell(s) may be combined with carcinoembionic antigen-related cell adhesion molecule 1 (CEACAM1); endothelin-B receptor (ET-B); or vascular endothelial growth factor inhibitors gravin/AKAP12, a scaffolding protein for protein kinase A and protein kinase C. See, e.g., Robert S. Korbel “Anti-angiogenic Therapy: A Universal Chemosensitization Strategy for Cancer?”, Science 26 May 2006, Vol 312, No. 5777:1171-1175.

Applicants note that the type of immune cells that may be modified as disclosed above is not particularly limiting. In some embodiments, a cell used for modification may be an immune cell used in adoptive immunotherapy. Non-limiting examples of such cells include autologous cells, allogenic cells, and precursor cells. In some embodiments, the cell to be modified may be a terminally differentiated effector cell. In some embodiments, the cell to be modified may be a T-cell, such as a monoclonal T-cell or a polyclonal T-cell. In some embodiments, the cell to be modified may be tumor-antigen specific T lymphocyte. In some embodiments, the cell to be modified may be a cytotoxic T-cell, such as an activated cytotoxic T-cell. In some embodiments, the cell to be modified may be a cytotoxic lymphocyte. In further embodiments, the cell to be modified may be an NK cell, a monocyte or a macrophage, such as a monocyte derived macrophage.

The above-described immune cells, as part of the human or animal immune system, may be capable of recognizing a site affected by a disease, such as a tumor site. Such immune cells may also have a natural capability to actively migrate during an inflammatory or anti-tumoral response in non-lymphatic tissues and to infiltrate a diseased site. Thus, such capabilities may be used to improve the targeting ability of a delivery system through surface modification of the delivery system with components (e.g., cellular membranes) isolated from the immune cells.

In sum, various cellular membranes that are derived from various cells may be isolated, characterized, and used for surface modification of the delivery systems of the present invention. In some embodiments, the cellular membranes are derived from immune cells, such as genetically modified immune cells. In some embodiments, the cellular membranes show an enhanced targeting ability. In some embodiments, it may be desirable to reproduce on the surface of the delivery system all the protein and lipid properties and functions of the immune cell that are necessary for migration and infiltration into a diseased tissue. As set forth below, various cell modification and isolation devices may be used to accomplish these tasks.

Cell Modification Devices

A person of ordinary skill in the art will also recognize that various cell modification devices may be used to modify cellular membranes. For instance, in some embodiments, cell modification devices may include a cell isolating component and a cell modification component.

The cell isolating component may be used for isolating cells (e.g., immune cells) from a biological sample (e.g., a blood sample). In some embodiments, the cell isolating component may also comprise a syringe.

Likewise, the cell modification component may be used for enhancing a targeting capability of the isolated cell. Thus, in some embodiments, the cell modification component may include a Sleeping Beauty system. Non-limiting examples of such systems are disclosed in the following references: Singh, H., et al., Cancer Res, 2008. 68 (8):2961-71; Frommolt, R. et al., 2006. 3 (3):345-349; Huang, X., et al., Blood. 2006. 107 (2): 483-491; and Hackett, P. B. et al., A Transposon and Transposase System for Human Application. Mol Ther, 2010.

In additional embodiments, the cell modification devices of the present invention may also include a holding or fixing component. Such components may be used to fix or hold the isolated cell.

Cellular Membrane Isolation Devices

A person of ordinary skill in the art will also recognize that various cellular membrane isolation devices may be used in the present invention. For instance, in some embodiments, the cellular membranes may be isolated and purified by ultracentrifugation through a discontinuous sucrose density gradient. In such embodiments, the visualized lipid fraction may be collected, purified, and characterized by various methods. Such methods may include qualitative and/or quantitative assays for proteins and/or lipids in order to selectively recover the fractions corresponding only to a cellular membrane of interest (e.g., a plasma cellular membrane). Thereafter, the selected cellular membranes may be used for the surface modification of a delivery system.

Methods of Making Delivery Systems

Additional embodiments of the present invention pertain to methods of making delivery systems. Such methods generally comprise: (1) isolating a cellular membrane from a cell; and (2) associating at least a portion of the isolated cellular membrane with a surface of a microparticle or a nanoparticle to form the delivery system (hereinafter “surface modification” or “modification”).

In some embodiments, the surface modification occurs by disposing the isolated cellular membrane on a surface of the microparticle or nanoparticle. In some embodiments, the disposing may occur by incubation. In additional embodiments, the methods may further include a step of obtaining the microparticle or nanoparticle from various sources (as discussed in more detail below). In further embodiments, the methods may comprise the loading of one or more active agents into the microparticle or nanoparticle prior to surface modification. In additional embodiments, the methods of the present invention may also include a step of disposing an adhesive agent on the surface of the microparticle or nanoparticle prior to surface modification. In further embodiments, the cellular membrane may be isolated from a source by ultracentrifugation through a discontinuous sucrose density gradient.

A person of ordinary skill in the art will also recognize that surface modification of delivery systems may be performed in a number of ways. A particular surface modification method may depend on various attributes of a particular delivery system. Such properties may include surface properties, such as the charge and the roughness of the delivery system.

In some embodiments, the surface modification of delivery systems may involve the surface modification of a pre-existing delivery system. In such cases, isolated cellular membranes may be incubated with the delivery systems. In some embodiments, the incubation temperature may be from 0° C. to 20° C., from 0° C. to 10° C., from 2° C. to 6° C., from 3° C. to 5° C., or 4° C.

The incubation times may also vary. In some embodiments, the incubation time may be at least 0.1 hour, at least 0.2 hour, at least 0.5 hour, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, or at least 15 hours. In more specific embodiments, the incubation time may be from 10-18 hours, or from 12 to 15 hours.

In some embodiments, the surface modification may involve forming from the isolated cellular membrane(s) lipid particle(s), such as multilamellar vesicle(s) or liposome(s). For example, the isolated cellular membrane may be placed into an aqueous solution or media so that hydrophobic interaction among lipid tails of the isolated cellular membranes would lead to a spontaneous formation of the lipid particle, such as a multilamellar vesicle(s). In some embodiments, the formed lipid particles may be extruded in order to obtain lipid particles, such as liposomes with a desired size.

In some embodiments, in order to load or encapsulate an active agent (e.g., a therapeutic and/or imaging agent) into the lipid particle (e.g., a liposome), the active agent may be added to the aqueous solution or media prior to or during the formation of the lipid particle. In some embodiments, the formed lipid particles may be incubated with a delivery system to allow the interaction, rupture and spreading of the lipid particles on the surface of the delivery system. The use of lipid particles made with the isolated cellular membranes may ensure the complete surface coverage of the delivery system with the isolated cellular membrane.

In some embodiments, a complete surface coverage of a delivery system with an isolated cellular membrane may be necessary. Without being bound by theory, it is envisioned that the complete surface coverage may delay the release of an active agent from the carrying delivery system. The complete surface coverage may also help avoid the interaction of the delivery system's surface with blood opsonization factors that may activate the immune response. The activation of the immune response may subsequently lead to the sequestration of the delivery system from the macrophages of the reticuloendothelial system (RES).

After being administered to a subject, the delivery system that was modified with isolated cellular membrane(s) from a particular cell (e.g., immune cells) may move and mimic “natural functions” of those cells within a body of the subject. The modified delivery system may also migrate to and accumulate at a site affected by a disease. At the disease site, the cellular membrane(s) may be dissolved by environmental factors, such as enzymes and pH. Thereafter, the load of the delivery system may be released. For example, when the disease site is a tumor site and the load of the delivery system includes a cytolytic or cytotoxic agent, the release of the load may exert a cytolytic or cytotoxic action on the tumor cells and thereby kill them.

In some embodiments, the release of the load from the modified delivery system may take from 1 to 30 days, from 2 to 21 days, from 3 to 14 days, or any time within these ranges. As set forth below, various delivery systems may be used with various embodiments of the present invention.

Delivery Systems

A person of ordinary skill in the art will also recognize that a number of delivery systems may be used in the present invention. Delivery systems of the present invention generally comprise a microparticle or a nanoparticle (hereinafter “particles”). In various embodiments, the microparticle or nanoparticle is at least one of multistage particles, porous particles, porous silicon particles, porous silica particles, non-porous particles, fabricated particles, polymeric particles, synthetic particles, semiconducting particles, viruses, gold particles, silver particles, quantum dots, indium phosphate particles, iron oxide particles, micelles, lipid particles, liposomes, silica particles, mesoporous silica particles, PLGA-based particles, gelatin-based particles, carbon nanotubes, fullerenes, and combinations thereof.

Generally, the surface of particles of the delivery systems of the present invention contain at least a portion of isolated cellular membranes. In addition, the particles of the delivery systems may be associated, loaded and/or encapsulated with one or more active agents. In some embodiments, the active agent is on a surface of the microparticle or nanoparticle. In other embodiments, the active agent is inside the microparticle or nanoparticle. In further embodiments, active agent is on a surface and inside a microparticle or nanoparticle. In the case of multistage delivery systems that will be described in more detail below, a second stage particle may contain the one or more active agents in some embodiments.

In various embodiments, the particles of the present invention may also have a functionalized surface. For instance, in various embodiments, a surface of a particle may be functionalized with functionalizing agents such as peptides, polymers, chitosans, contrasting agents, imaging agents and calcium phosphates. In more specific embodiments, a surface of a particle may be functionalized with a polymer that becomes swellable in response to a stimulus (e.g., change in temperature, change in pH, change in pressure, and combinations thereof).

In some embodiments, the microparticle or nanoparticle comprises a lipid particle with a lipid layer, such as a liposome. In some embodiments, the lipid layer or liposome comprises at least a portion of an isolated cellular membrane.

In some embodiments, the delivery system may be a liposome, such as a unilamellar liposome, or a multilamellar liposome. In some embodiments, the delivery system may comprise a polymer. For example, in some embodiments, the delivery system may comprise a polysaccharide, such as chitosan or agarose. Yet, in some embodiments, the delivery system may comprise polyethyleneimine.

In some embodiments, the delivery system may comprise a microparticle or nanoparticle (hereinafter “particles” or “particle”). In some embodiments, the particles may be man-made or fabricated (i.e., non-natural microparticles or nanoparticles). In some embodiments, the particles may be pre-existing particles.

In some embodiments, the particle may be a porous particle (i.e., a particle that comprises a porous material). In some embodiments, the porous material may be a porous oxide material or a porous etched material. Examples of porous oxide materials include, but are not limited to, porous silicon oxide, porous aluminum oxide, porous titanium oxide and porous iron oxide.

The term “porous etched materials” refers to a material in which pores are introduced via a wet etching technique, such as electrochemical etching or electroless etching. Examples of porous etched materials include porous semiconductor materials, such as porous silicon, porous germanium, porous GaAs, porous InP, porous SiC, porous SiₓGe₁₋ₓ, porous GaP, and porous GaN. Methods of making porous etched particles are disclosed, for example, in US Patent Application Publication No. 2008/0280140.

In some embodiments, the porous particle may be a nanoporous particle. In some embodiments, an average pore size of the porous particle may be from about 1 nm to about 1 micron, from about 1 nm to about 800 nm, from about 1 nm to about 500 nm, from about 1 nm to about 300 nm, from about 1 nm to about 200 nm, or from about 2 nm to about 100 nm. In some embodiments, the average pore size of the porous particle can be no more than 1 micron, no more than 800 nm, no more than 500 nm, no more than 300 nm, no more than 200 nm, no more than 100 nm, no more than 80 nm, or no more than 50 nm. In some embodiments, the average pore size of the porous particle can be from about 5 nm to about 100 nm, from about 10 nm to about 60 nm, from about 20 nm to about 40 nm, or from about 10 nm to about 30 nm.

In some embodiments, the average pore size of the porous particle can be from about 1 nm to about 10 nm, from about 3 nm to about 10 nm, or from about 3 nm to about 7 nm. In general, pores sizes may be determined using a number of techniques, including N₂ adsorption/desorption and microscopy, such as scanning electron microscopy.

In some embodiments, pores of the porous particle may be linear pores. Yet, in some embodiments, pores of the porous particle may be sponge-like pores. When the isolated cellular membrane is disposed on a surface of a porous particle, an active agent, such as a therapeutic and/or imaging agent, may be loaded into pores of the porous particle. Such loading may occur prior to or during the surface modification of the particle with an isolated cellular membrane. Methods of loading active agents into porous particles are disclosed, for example, in U.S. Pat. No. 6,107,102 and US Patent Application Publication No. 2008/0311182. In some embodiments, after the active agent is loaded, the pores of the porous particle may be sealed or capped prior to the disposal of the isolated cellular membrane on the particle. In some embodiments, the isolated cellular membrane disposed on a surface of the particle may be used for sealing and/or capping the load within the porous particle.

In some embodiments, at least a portion of the porous particle may comprise a biodegradable region. In many embodiments, the whole particle may be biodegradable.

In general, porous silicon may be bioinert, bioactive or biodegradable depending on its porosity and pore size. Also, a rate or speed of biodegradation of porous silicon may depend on its porosity and pore size. See, e.g., Canham, Biomedical Applications of Silicon, in Canham LT, editor. Properties of porous silicon. EMIS Data Review Series No. 18. London: INSPEC. PP. 371-376. The biodegradation rate may also depend on surface modification. Porous silicon particles and methods of their fabrication are disclosed, for example, in the following references: Cohen M. H. et al., Biomedical Micro-devices 5:3, 253-259, 2003; US Patent Application Publication No. 2003/0114366; U.S. Pat. Nos. 6,107,102 and 6,355,270; US Patent Application Publication No. 2008/0280140; PCT Publication No. WO 2008/021908; Foraker, A. B. et al. Pharma. Res. 20 (1), 110-116 (2003); and Salonen, J. et al. Jour. Contr. Rel. 108, 362-374 (2005). In addition, porous silicon oxide particles and methods of their fabrication are disclosed, for example, in Paik J. A. et al., J. Mater. Res., Vol 17, August 2002, p. 2121.

In some embodiments, the particle may comprise a biodegradable material. For oral administration, such material may be a material designed to erode in the GI tract. In some embodiments, the biodegradable particle may be formed of a metal, such as iron, titanium, gold, silver, platinum, copper, alloys and oxides thereof. In some embodiments, the biodegradable material may be a biodegradable polymer, such as polyorthoesters, polyanhydrides, polyamides, polyalkylcyanoacrylates, polyphosphazenes, and polyesters. Exemplary biodegradable polymers are described, for example, in U.S. Pat. Nos. 4,933,185, 4,888,176, and 5,010,167. Specific examples of such biodegradable polymer materials include poly(lactic acid), polyglycolic acid, polyglycolic-lactic acid (PGLA); polycaprolactone, polyhydroxybutyrate, poly(N-palmitoyl-trans-4-hydroxy-L-proline ester) and poly(DTH carbonate).

The particles may also have a variety of shapes and sizes. However, the dimensions of such particles are not particularly limited and may depend on a particular application. For example, for intravascular administration, a maximum characteristic size of the particle may be smaller than a radius of the smallest capillary in a subject, which is about 4 to 5 microns for humans. In some embodiments, the maximum characteristic size of the particles may be less than about 100 microns, less than about 50 microns, less than about 20 microns, less than about 10 microns, less than about 5 microns, less than about 4 microns, less than about 3 microns, less than about 2 microns, or less than about 1 micron. Yet, in some embodiments, the maximum characteristic size of the particle may be from 100 nm to 3 microns, from 200 nm to 3 microns, from 500 nm to 3 microns, or from 700 nm to 2 microns. In additional embodiments, the maximum characteristic size of the particle may be greater than about 2 microns, greater than about 5 microns, or greater than about 10 microns.

In addition, the shape of particles are not particularly limited. In some embodiments, the particle may be a spherical particle. Yet, in some embodiments, the particle may be a non-spherical particle. In some embodiments, the particle can have a symmetrical shape. Yet, in some embodiments, the particle may have an asymmetrical shape. In other embodiments, the particle may have a selected non-spherical shape configured to facilitate a contact between the particle and a surface of the target site, such as an endothelium surface of the inflamed vasculature. Examples of appropriate shapes include, but are not limited to, an oblate spheroid, a disc, or a cylinder.

In other embodiments, the particles may have a surface that contains at least a portion of an isolated cellular membrane. Such portions may cover the entire surface or part of the surface. In more specific embodiments, the particles may be such that only a portion of its surface defines a shape configured to facilitate a contact between the particle and a surface of the target site, such as an endothelium surface. For example, the particle can be a truncated oblate spheroidal particle. The dimensions and shapes of particles that may facilitate a contact between the particle and a surface of the target site may be evaluated using methods disclosed in US Patent Application Publications Nos. 2008/0206344 and 2010/0029785.

In some embodiments, the particles may be such that a release of the load may take place at a time after administering the system to a subject. In some embodiments, the post-administration release may take place at least one hour, at least 2 hours, at least 4 hours, at least 6 hours, at least 8 hours, at least 10 hours, at least 12 hours, at least 18 hours, at least 1 day, at least 2 days, at least 3 days, at least 4 days, at least 5 days, at least 6 days, at least 7 days, at least 8 days, at least 9 days, or at least 10 days after administering the system to a subject.

The particle on which the isolated cellular membrane may be disposed may be prepared using a number of techniques. In some embodiments, the particle of the delivery system may be a particle produced utilizing a top-down microfabrication or nanofabrication technique. Such techniques include, without limitation: photolithography, electron beam lithography, X-ray lithography, deep UV lithography, nanoimprint lithography, and dip pen nanolithography. Such fabrication methods may allow for a scaled up production of particles that are uniform or substantially identical in dimensions.

In some embodiments, the delivery system may be a multistage delivery system. Such delivery systems may comprise a larger first stage microparticle or nanoparticle that may contain one or more smaller size second stage particles. Multistage delivery systems are disclosed, for example, in the following references: US Patent Application Publications Nos. 2008/0311182 and 2008/0280140; and Tasciotti E. et al, 2008 Nature Nanotechnology 3, 151-157. In case of the multistage delivery system, the isolated cellular membrane may be used for modifying a surface of the first stage particle.

In many embodiments, the first stage particle of the multistage delivery object may already contain one or more second stage particles when the isolated cellular membrane is disposed on the first stage particle. For example, when the first stage particle is a porous particle, its pores may be loaded with one or more second stage particles prior to the surface modification with the isolated cellular membrane. After the second stage particles are loaded, the pores of the porous first stage particle may be sealed or capped prior to the disposal of the isolated cellular membrane on the first stage particle. In some embodiments, the isolated cellular membrane disposed on a surface of the particle may be used for sealing and/or capping the second stage particles within the porous particle.

Additional delivery systems that may be used with various embodiments of the present invention are disclosed in the following references: PCT Publications Nos. WO 2008/041970 and WO 2008/021908; U.S. Patent Application Publications Nos. 2008/0102030, 2003/0114366, 2008/0206344, 2008/0280140, 2010/0029785, and 2008/0311182; PCT Patent Application Nos. PCT/US2008/014001 (filed on Dec. 23, 2008), PCT/US2009/000239 (filed on Jan. 15, 2009), and PCT/US11/27746 (filed on Mar. 9, 2011); and U.S. Pat. Nos. 6,107,102 and 6,355,270.

In some embodiments, a surface of a pre-existing or fabricated particle may be modified in order to facilitate adhesion of the isolated cellular membrane. For example, in some embodiments, an adhesive agent or molecule may be disposed on the surface of a pre-existing particle prior to adhesion of the isolated cellular membrane. In some embodiments, such an adhesive agent may be a thiol-containing molecule or an amino group containing molecule. For a pre-existing particle, which has an oxide containing surface, such as silicon or silica particles, the adhesive agent may be silane, such as an aminosilane (e.g., 3-aminopropyltriethoxysilane) or a thiol-containing silane (e.g., 3-mereaptopropyltrimethoxysilane).

Active Agents

A person of ordinary skill in the art will also recognize that various active agents may be used in the present invention. In various embodiments, the active agent may be a therapeutic agent, an imaging agent or a combination thereof. In some embodiments, the selection of the active agent may depend on a desired application. Non-limiting examples of active agents are described below.

Therapeutic Agents

A therapeutic agent may be a physiologically or pharmacologically active substance that can produce a desired biological effect in a targeted site in an animal, such as a mammal or a human. The therapeutic agent may be any inorganic or organic compound. Examples include, without limitation, peptides, proteins, nucleic acids (including siRNA, miRNA and DNA), polymers, and small molecules. In various embodiments, the therapeutic agents may be characterized or uncharacterized.

Therapeutic agents of the present invention may also be in various forms. Such forms include, without limitation, unchanged molecules, molecular complexes, and pharmacologically acceptable salts (e.g., hydrochloride, hydrobromide, sulfate, laurate, palmitate, phosphate, nitrite, nitrate, borate, acetate, maleate, tartrate, oleate, salicylate, and the like). For acidic therapeutic agents, salts of metals, amines or organic cations (e.g., quaternary ammonium) can be used in some embodiments. Derivatives of drugs, such as bases, esters and amides can also be used as a therapeutic agent. A therapeutic agent that is water insoluble can be used in a form that is a water soluble derivative thereof, such as a base derivative. In such instances, the derivative therapeutic agent may be converted to the original therapeutically active form upon delivery to a targeted site. Such conversions can occur by various metabolic processes, including enzymatic cleavage, hydrolysis by the body pH, or by other similar processes.

Non-limiting examples of therapeutic agents include anti-inflammatory agents, anti-cancer agents, anti-proliferative agents, anti-vascularization agents, wound repair agents, tissue repair agents, thermal therapy agents, and combinations thereof.

More specific but non-limiting examples of therapeutic agents include anti-cancer agents, such as anti-proliferative agents and anti-vascularization agents; antimalarial agents; OTC drugs, such as antipyretics, anesthetics and cough suppressants; antiinfective agents; antiparasites, such as anti-malaria agents (e.g., Dihydroartemisin); antibiotics, such as penicillins, cephalosporins, macrolids, tetracyclines, aminglycosides, and anti-tuberculosis agents; antifungal/antimycotic agents; genetic molecules, such as anti-sense oligonucleotides, nucleic acids, oligonucleotides, DNA, RNA; anti-protozoal agents; antiviral agents, such as acyclovir, gancyclovir, ribavirin, anti-HIV agents, and anti-hepatitis agents; anti-inflammatory agents, such as NSAIDs, steroidal agents, cannabinoids; anti-allergic agents, such as antihistamines, (e.g., fexofenadine); bronchodilators; vaccines or immunogenic agents, such as tetanus toxoid, reduced diphtheria toxoid, acellular pertussis vaccine, mums vaccine, smallpox vaccine, anti-HIV vaccines, hepatitis vaccines, pneumonia vaccines and influenza vaccines; anesthetics, including local anesthetics; antipyretics, such as paracetamol, ibuprofen, diclofenac, aspirin; agents for treatment of severe events, such as cardiovascular attacks, seizures, hypoglycemia; anti-nausea and anti-vomiting agents; immunomodulators and immunostimulators; cardiovascular drugs, such as beta-blockers, alpha-blockers and calcium channel blockers; peptide and steroid hormones, such as insulin, insulin derivatives, insulin detemir, insulin monomeric, oxytocin, LHRH, LHRH analogues, adreno-corticotropic hormone, somatropin, leuprolide, calcitonin, parathyroid hormone, estrogens, testosterone, adrenal corticosteroids, megestrol, progesterone, sex hormones, growth hormones and growth factors; peptide and protein related drugs, such as amino acids, peptides, polypeptides and proteins; vitamins, such as Vitamin A, vitamins from the Vitamin B group, folic acid, Vitamin C, Vitamin D, Vitamin E, Vitamin K, niacin, and derivatives of Vitamins A-E; autonomic nervous system drugs; fertilizing agents; antidepressants, such as buspirone, venlafaxine, benzodiazepins, selective serotonin reuptake inhibitors (SSRIs), sertraline, citalopram, tricyclic antidepressants, paroxetine, trazodone, lithium, bupropion, sertraline, and fluoxetine; agents for smoking cessation, such as bupropion and nicotine; lipid-lowering agents, such as inhibitors of 3 hydroxy-3-methylglutaryl-coenzyme A (HMG-CoA) reductase, simvastatin and atrovastatinl; agents for CNS or spinal cord, such as benzodiazepines, lorazepam, hydromorphone, midazolam, Acetaminophen, 4′-hydroxyacetanilide, barbiturates and anesthetics; anti-epilepsic agents, such as valproic acid and its derivatives and carbamazepin; angiotensin antagonists, such as valsartan; anti-psychotic agents and anti-schizophrenic agents, such as quetiapine and risperidone; agents for treatment of Parkinsonian syndrome, such as L-dopa and its derivatives and trihexyphenidyl; anti-Alzheimer agents, such as cholinesterase inhibitors, galantamine, rivastigmine, donepezil, tacrine, memantine and N-methyl D-aspartate (NMDA) antagonists; agents for treatment of non-insulin dependent diabetes, such as metformine; agents for treatment of erectile dysfunction, such as sildenafil, tadalafil, papaverine, vardenafil and PGE1; prostaglandins; agents for bladder dysfunction, such as oxybutynin, propantheline bromide, trospium and solifenacin succinate; agents for treatment menopausal syndrome, such as estrogens, non-estrogen compounds and agents for treatment hot flashes in postmenopausal women; agents for treatment of primary or secondary hypogonadism, such as testosterone; cytokines, such as TNF, interferons, IFN-α, IFN-β, interleukins; CNS stimulants; muscle relaxants; anti-paralytic gas agents; narcotics and antagonists, such as opiates and oxycodone; painkillers, such as opiates, endorphins, tramadol, codein, NSAIDs and gabapentine; hypnotics, such as zolpidem, benzodiazepins, barbiturates and ramelteon; histamines and antihistamines; antimigraine drugs, such as imipramine, propranolol and sumatriptan; diagnostic agents, such as phenolsulfonphthalein, Dye T-1824, vital dyes, potassium ferrocyanide, secretin, pentagastrin and cerulein; topical decongestants or anti-inflammatory agents; anti-acne agents, such as retinoic acid derivatives, doxicillin and minocyclin; ADHD related agents, such as methylphenidate, dexmethylphenidate, dextroamphetamine, d- and l-amphetamin racemic mixture and pemoline; diuretic agents; anti-osteoporotic agents, such as bisphosphonates, aledronate, pamidronate and tirphostins; osteogenic agents; anti-asthma agents; anti-spasmotic agents, such as papaverine; agents for treatment of multiple sclerosis and other neurodegenerative disorders, such as mitoxantrone, glatiramer acetate, interferon β-1α, interferon β-1β; and plant derived agents from leaves, roots, flowers, seeds, stems or branches extracts.

In additional embodiments, the therapeutic agents of the present invention can also be chemotherapeutic agents, immunosuppressive agents, cytokines, cytotoxic agents, nucleolytic compounds, radioactive isotopes, receptors, and pro-drug activating enzymes. The therapeutic agents of the present invention may be naturally occurring or produced by synthetic or recombinant methods, or any combination thereof.

In various embodiments, drugs that are affected by classical multidrug resistance can have particular utility as therapeutic agents in the present invention. Such drugs include, without limitation, vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel).

In additional embodiments, the therapeutic agent may be a cancer chemotherapy agent. Examples of suitable cancer chemotherapy agents include, without limitation: nitrogen mustards, nitrosorueas, ethyleneimine, alkane sulfonates, tetrazine, platinum compounds, pyrimidine analogs, purine analogs, antimetabolites, folate analogs, anthracyclines, taxanes, vinca alkaloids, and topoisomerase inhibitors and hormonal agents. Additional exemplary chemotherapy drugs that may be used as therapeutic agents in the present invention include, without limitation: Actinomycin-D, Alkeran, Ara-C, Anastrozole, Asparaginase, BiCNU, Bicalutamide, Bleomycin, Busulfan, Capecitabine, Carboplatin, Carboplatinum, Carmustine, CCNU, Chlorambucil, Cisplatin, Cladribine, CPT-11, Cyclophosphamide, Cytarabine, Cytosine arabinoside, Cytoxan, Dacarbazine, Dactinomycin, Daunorubicin, Dexrazoxane, Docetaxel, Doxorubicin, DTIC, Epirubicin, Ethyleneimine, Etoposide, Floxuridine, Fludarabine, Fluorouracil, Flutamide, Fotemustine, Gemcitabine, Herceptin, Hexamethylamine, Hydroxyurea, Idarubicin, Ifosfamide, Irinotecan, Lomustine, Mechlorethamine, Melphalan, Mercaptopurine, Methotrexate, Mitomycin, Mitotane, Mitoxantrone, Oxaliplatin, Paclitaxel, Pamidronate, Pentostatin, Plicamycin, Procarbazine, Rituximab, Steroids, Streptozocin, STI-571, Streptozocin, Tamoxifen, Temozolomide, Teniposide, Tetrazine, Thioguanine, Thiotepa, Tomudex, Topotecan, Treosulphan, Trimetrexate, Vinblastine, Vincristine, Vindesine, Vinorelbine, VP-16, Xeloda, and Camptothecin.

Additional cancer chemotherapy drugs that may be used as therapeutic agents in the present invention include, without limitation: alkylating agents, such as Thiotepa and cyclosphosphamide; alkyl sulfonates, such as Busulfan, Improsulfan and Piposulfan; aziridines, such as Benzodopa, Carboquone, Meturedopa, and Uredopa; ethylenimines and methylamelamines, including altretamine, triethylenemelamine, trietylenephosphoramide, triethylenethiophosphaoramide and trimethylolomelamine; nitrogen mustards such as Chlorambucil, Chlomaphazine, Cholophosphamide, Estramustine, Ifosfamide, mechlorethamine, mechlorethamine oxide hydrochloride, Melphalan, Novembiehin, Phenesterine, Prednimustine, Trofosfamide, and uracil mustard; nitroureas, such as Cannustine, Chlorozotocin, Fotemustine, Lomustine, Nimustine, and Ranimustine; antibiotics, such as Aclacinomysins, Actinomycin, Authramycin, Azaserine, Bleomycins, Cactinomycin, Calicheamicin, Carabicin, Caminomycin, Carzinophilin, Chromoinycins, Dactinomycin, Daunorubicin, Detorubicin, 6-diazo-5-oxo-L-norleucine, Doxorubicin, Epirubicin, Esorubicin, Idambicin, Marcellomycin, Mitomycins, mycophenolic acid, Nogalamycin, Olivomycins, Peplomycin, Potfiromycin, Puromycin, Quelamycin, Rodorubicin, Streptonigrin, Streptozocin, Tubercidin, Ubenimex, Zinostatin, and Zorubicin; anti-metabolites, such as Methotrexate and 5-fluorouracil (5-FU); folic acid analogues, such as Denopterin, Methotrexate, Pteropterin, and Trimetrexate; purine analogs, such as Fludarabine, 6-mercaptopurine, Thiamiprine, and Thioguanine; pyrimidine analogs, such as Ancitabine, Azacitidine, 6-azauridine, Carmofur, Cytarabine, Dideoxyuridine, Doxifluridine, Enocitabine, Floxuridine, and 5-FU; androgens, such as Calusterone, Dromostanolone, Propionate, Epitiostanol, Rnepitiostane, and Testolactone; anti-adrenals, such as aminoglutethimide, Mitotane, and Trilostane; folic acid replenisher, such as frolinic acid; aceglatone; aldophosphamide glycoside; aminolevulinic acid; Amsacrine; Bestrabucil; Bisantrene; Edatraxate; Defofamine; Demecolcine; Diaziquone; Elformithine; elliptinium acetate; Etoglucid; gallium nitrate; hydroxyurea; Lentinan; Lonidamine; Mitoguazone; Mitoxantrone; Mopidamol; Nitracrine; Pentostatin; Phenamet; Pirarubicin; podophyllinic acid; 2-ethylhydrazide; Procarbazine; PSK®; Razoxane; Sizofrran; Spirogermanium; tenuazonic acid; triaziquone; 2,2′,2″-trichlorotriethylamine; Urethan; Vindesine; Dacarbazine; Mannomustine; Mitobronitol; Mitolactol; Pipobroman; Gacytosine; Arabinoside (“Ara-C”); cyclophosphamide; thiotEPa; taxoids, e.g., Paclitaxel (Taxol®, Bristol-Myers Squibb Oncology, Princeton, N.J.) and Doxetaxel (Taxotere®, Rhone-Poulenc Rorer, Antony, France); Chlorambucil; Gemcitabine; 6-thioguanine; Mercaptopurine; Methotrexate; platinum analogs such as Cisplatin and Carboplatin; Vinblastine; platinum; etoposide (VP-16); Ifosfamide; Mitomycin C; Mitoxantrone; Vincristine; Vinorelbine; Navelbine; Novantrone; Teniposide; Daunomycin; Aminopterin; Xeloda; Ibandronate; CPT-11; topoisomerase inhibitor RFS 2000; difluoromethylomithine (DMFO); retinoic acid; Esperamicins; Capecitabine; and pharmaceutically acceptable salts, acids or derivatives of any of the above.

Additional therapeutic agents that are suitable for use in the present invention include, without limitation, anti-hormonal agents that act to regulate or inhibit hormone action on tumors. Non-limiting examples of such anti-hormonal agents include anti-estrogens, including for example Tamoxifen, Raloxifene, aromatase inhibiting 4(5)-imidazoles, 4 Hydroxytamoxifen, Trioxifene, Keoxifene, Onapristone, and Toremifene (Fareston); anti-androgens, such as Flutamide, Nilutamide, Bicalutamide, Leuprolide, and Goserelin; and pharmaceutically acceptable salts, acids or derivatives of any of the above.

In additional embodiments of the present invention, cytokines can be also used as therapeutic agents. Non-limiting examples of such cytokines are lymphokines, monokines, and traditional polypeptide hormones. Additional examples include growth hormones, such as human growth hormone, N-methionyl human growth hormone, and bovine growth hormone; parathyroid hormone; thyroxine; insulin; proinsulin; relaxin; prorelaxin; glycoprotein hormones, such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet growth factor; transforming growth factors (TGFs) such as TGF-α and TGF-β; insulin-like growth factor-I and -II; erythropoietin (EPO); osteoinductive factors; interferons, such as interferon-α, -β and -γ; colony stimulating factors (CSFs), such as macrophage-CSF (M-CSF), granulocyte-macrophage-CSF (GM-CSF), and granulocyte-CSF (GCSF); interleukins (ILs), such as IL-1, IL-1α, IL-2, IL-3, IL-4, IL-5, IL-6, IL-7, IL-8, IL-9, IL-11, IL-12, IL-15; tumor necrosis factors, such as TNF-α or TNF-β; and other polypeptide factors, including LIF and kit ligand (KL). As used herein, the term cytokine includes proteins from natural sources or from recombinant sources (e.g., from T-cell cultures and biologically active equivalents of the native sequence cytokines).

In some embodiments, the therapeutic agent can also be an antibody-based therapeutic agent, such as Herceptin, Erbitux, Avastin, Rituxan, Panitumumab, Mylotarg, Zenapax, Simulect, Enbrel, Adalimumab, and Remicade.

In some embodiments, the therapeutic agent can be a nanoparticle. For example, in some embodiments, the nanoparticle can be a nanoparticle that can be used for a thermal ablation or a thermal therapy. Examples of such nanoparticles include any metal and semiconductor based nanoparticle, which includes but is not limited to: iron oxide, quantum dots (both CdSe and indium phosphate), gold (spherical, rods, hollow nanoshperes), silver, carbon nanotubes, carbon fullerenes, silica, and silicon nanoparticles.

Imaging Agents

Imaging agents in the present invention may be substances that provide imaging information about a targeted site in a body of an animal, such as a mammal or a human being. In some embodiments, the imaging agent may comprise a magnetic material, such as iron oxide or a gadolinium containing compound. In additional embodiments, such imaging agents may be utilized for magnetic resonance imaging (MRI).

For embodiments involving optical imaging, the imaging agent may be, for example, semiconductor nanocrystals or quantum dots. For optical coherence tomography imaging, the imaging agent may be a metal, such as gold or silver nanocage particles. In some embodiments, the imaging agent may be metal nanoparticles, such as gold or silver nanoparticles. In additional embodiments, the imaging agents may be semiconductor nanoparticles, such as quantum dots.

In some embodiments, the imaging agent may be an ultrasound contrast agent, such as a microbubble, a nanobubble, an iron oxide microparticle, or an iron oxide nanoparticle. In some embodiments, the imaging agent may be a molecular imaging agent that can be covalently or non-covalently attached to a particle's surface.

In some embodiments, the imaging agent may be a metal ion complex/conjugate that can be covalently or non-covalently attached to a particle's surface. In some embodiments, the imaging agent may be a radionucleotide that can be covalently or non-covalently attached to a particle's surface.

Modes of Administration

Additional embodiments of the present invention pertain to methods of administering the delivery systems of the present invention to a subject. In some embodiments, the delivery systems of the present invention may be administered as part of a therapeutic composition that includes a plurality of delivery systems. In some embodiments, the delivery systems may be administered to a subject, such as a human. In more specific embodiments, the delivery systems may be administered to a human being suffering from a condition associated with inflammation, such as cancer. In further embodiments the delivery systems migrate to a site associated with the condition (i.e., inflammation or cancer) within the subject after administration. Thereafter, the active agent is released from delivery system after migration to the site.

A person of ordinary skill in the art will also recognize that various suitable administration methods may be used to treat, prevent, diagnose and/or monitor a physiological condition, such as a disease. The particular administration method employed for a specific application may be determined by the attending physician. Typically, the delivery systems of the present invention may be administered by one of the following routes: topical, parenteral, inhalation/pulmonary, oral, intraocular, intranasal, bucal, vaginal and anal. Non-limiting examples of parenteral administration may include intravenous administration (i.v.), intramuscular administration (i.m.) and subcutaneous (s.c.) injection. Additional modes of administration may also be envisioned by persons of ordinary skill in the art.

In addition, the delivery systems of the present invention may be administered systemically or locally. For instance, the non-parenteral examples of administration recited above are examples of local administration. Intravascular administration can be either local or systemic. In a specific example, local intravascular delivery can be used to bring a therapeutic substance to the vicinity of a known lesion by use of a guided catheter system, such as a CAT-scan guided catheter or portal vein injection. General injections, such as a bolus i.v. injection or continuous/trickle-feed i.v. infusion, are typically systemic.

In some embodiments, the composition containing the delivery system may be administered via i.v. infusion, intraductal administration, or via an intratumoral route.

Furthermore, the delivery systems of the present invention may be formulated as a suspension that contains a plurality of delivery systems. In some embodiments, individual delivery systems may be uniform in their dimensions and their content. To form the suspension, the delivery systems may be suspended in a suitable aqueous carrier vehicle. A suitable pharmaceutical carrier may the one that is non-toxic to the recipient at the dosages and concentrations employed. In addition, pharmaceutical carriers are desirably compatible with other ingredients in the formulation. Preparation of suspension of microfabricated particles is disclosed, for example, in US Patent Application Publication No. 2003/0114366.

Applications

A person of ordinary skill in the art will also recognize that the delivery systems of the present invention can be used for various purposes. For instance, in some embodiments, the delivery systems of the present invention may be used as systems for the delivery of an active agent, such as a therapeutic and/or imaging agent, to an animal. In many embodiments, the animal may be a warm blooded animal, such as a bird or a mammal. In certain embodiments, the animal may be a human being.

The delivery systems of the present invention may be used for treating, monitoring, preventing and/or diagnosing a number of diseases and conditions (e.g., inflammation, such as inflammation associated with cancer). In some embodiments, the delivery systems of the present invention may be particularly useful for oncological applications, such as for the treatment, monitoring, prevention and/or diagnosis of a cancerous condition (e.g., a tumor associated with cancer). In such embodiments, the delivery systems of the present invention may be used for delivering an active agent (e.g., a therapeutic and/or an imaging agent) to a site affected with cancer (e.g., a tumor site). Non-limiting examples of cancerous conditions that may be treated, monitored, prevented and/or diagnosed include, without limitation, lymphoma, colon cancer, lung cancer, pancreatic cancer, ovarian cancer, breast cancer and brain cancer.

In additional embodiments, the delivery systems of the present invention may be used to deliver an active agent to virus-infected cells. Thus, in such embodiments, the delivery systems of the present invention may be used for treating, monitoring, preventing and/or diagnosing viral infections.

In some embodiments, the delivery systems of the present invention may be used for targeting an inflamed site in a subject, such as an animal Therefore, in such embodiments, the delivery systems of the present invention may be used for treating, preventing, monitoring and/or diagnosing a condition or disease associated with an inflammation. Examples of such conditions include, without limitation: allergies; asthma; Alzheimer's disease; diabetes; hormonal imbalances; autoimmune diseases, such as rheumatoid arthritis and psoriasis; osteoarthritis; osteoporosis; atherosclerosis, including coronary artery disease; vasculitis; chronic inflammatory conditions, such as obesity; ulcers, such as Marjolin's ulcer; respiratory inflammations caused by asbestos or cigarette smoke; foreskin inflammations; inflammations caused by viruses, such as Human papilloma virus, Hepatitic B or C or Ebstein-Ban virus; Schistosomiasis; pelvic inflammatory disease; ovarian epitheal inflammation; Barrett's metaplasia; H. pylori gastritis; chronic pancreatitis; Chinese liver fluke infestation; chronic cholecystitis and inflammatory bowel disease; inflammation-associated cancers, such as prostate cancer, colon cancer, breast cancer; gastrointestinal tract cancers, such as gastric cancer, hepatocellular carcinoma, colorectal cancer, pancreatic cancer, gastric cancer, nasopharyngeal cancer, esophageal cancer, cholangiocarcinoma, gall bladder cancer and anogenital cancer; intergumentary cancer, such as skin carcinoma; respiratory tract cancers, such as bronchial cancer and mesothelioma; genitourinary tract cancer, such as phimosis, penile carcinoma and bladder cancer; and reproductive system cancer, such as ovarian cancer. Additional examples of conditions and diseases associated with inflammation that can be treated, prevented, diagnosed and/or monitored with the delivery systems of the present invention are disclosed in the following references: (1) M. Macarthur et al. Am. J. Physiol Gastrointest Livel Physiol. 286:G515-520, 2004; (2) Calogero et al. Breast Cancer Research, v. 9 (4), 2007; (3) Wienberg et al. J. Clin. Invest, 112: 1796-1808, 2003; and (4) Xu et. al., J. Clin Invest, 112:1821-1830, 2003.

### ADVANTAGES

The methods and systems of the present invention have numerous advantages over the methods and systems of the prior art. By way of background, methods for medical treatment using active agents have been known for a long time. However, in most of the prior art methods, the active agent was usually delivered to the whole human or animal body, without being targeted to a particular site affected by a disease. Thus, in the prior art methods, the active agent usually got distributed uniformly in the whole human or animal organism. Thus, one disadvantage of the prior art methods is that unaffected regions of the human or animal body can also be affected by the active agent. Furthermore, only a small part of the active agent could act in the diseased site.

In contrast, the delivery systems of the present invention allow for the delivery of an active agent preferentially to a diseased site. Such a targeted delivery may enhance the efficacy of the active agent. Such a targeted delivery may also allow one to avoid high doses of an active agent. This may in turn help prevent toxic side effects that are associated with the administration of high doses of various active agents.

The present invention also provides methods and devices that permit the modification of delivery systems with cellular membranes from various types of cells (e.g., immune cells). Thus, when reintroduced into the human or animal body, the modified delivery systems may be delivered to a desired body part or cells to exert a therapeutic and/or diagnostic effect there. By modifying the delivery systems in this way, it may be possible to treat or detect diseases with low doses of an active agent in a targeted manner (or to build up and strengthen a tissue in a targeted manner) without affecting uninvolved regions of the body.

In various embodiments, the delivery systems of the present invention may also provide at least one of the following advantages: (1) reduction of sequestration from the macrophages of the reticuloendothelial system (RES); (2) reduction of the immune system response; (3) increase circulation lifetime of the system; (4) provide specific and enhanced targeting of the diseased site; (5) increase therapeutic and/or monitoring effects at the diseased tissue. The above-described advantages may also become more apparent if the methods of the present invention are combined with existing adoptive and/or cellular immunotherapies.

In additional embodiments, the delivery systems of the present invention may also provide at least one of the following additional advantages: (1) increase the circulation time of the delivery system and reduce or prevent RES uptake of the delivery system by shielding the delivery system with the cellular membranes of leukocytes, such as autologous leukocytes; (2) prevent the release of the delivery system's load before the delivery system reaches a target site; (3) reduce a response of the immune system against the delivery system when it is introduced in a body of a recipient; (4) increase the transcytosis of the delivery system through the endothelial barrier in the vasculature of the recipient; (5) increase the accumulation of the delivery system at a diseased site, such as a tumor site; and (6) allow the delivery system to reduce or avoid internalization in lysosomes and/or endosomes in the body of the recipient.

The above-mentioned advantages may become more apparent in embodiments where an artificial delivery system is modified with a cellular membrane that is isolated from an immune cell. In such cases, the modification may “humanize” the artificial system by making it more compatible with the immune system of the recipient. Embodiments described herein are further illustrated by, though in no way limited to, the following working examples.

### EXAMPLES

### Example 1

### Modification of Delivery Systems with T-Cell Membranes

The endothelial barrier may play a fundamental role in controlling the transport of agents from the blood stream to the surrounding tissues. During an inflammatory event, peripheral blood cells (lymphocytes, monocytes and eosinophils) are recruited through a transendothelial migration (TEM) process. T cells can cross the endothelial wall through paracellular and transcellular routes following a controlled multistep progression that is closely regulated by localized adhesion molecules expressed on the endothelium (12, for references in round brackets see REFERENCES LIST 1 below).

Independently of the route taken, T-cell TEM may be triggered by the interaction between the intercellular adhesion molecule-1 (ICAM-1) on the endothelial membrane and the lymphocyte function-associated antigen-1 (LFA-1) on the T cell membrane. Interaction with ICAM-1 may trigger the activation of endothelial intracellular signaling pathways that result in extensive cytoskeletal remodeling events that alter endothelial cell contractility and function, facilitating leukocyte diapedesis. During TEM, endothelial cuplike structures enriched in ICAM-1 and LFA-1 surround the site of diapedesis and allow the leukocytes to squeeze through the tight junctions, as they migrate towards the interstitial tumor space (13, 14). Significantly, TEM may not require any molecular activation in the T cell aside from the remodeling of the cytoskeleton to fit the channel that is formed in the endothelial cell. Therefore, TEM may occur upon contact with a T cell membrane and may not require an active participation of the T cell. The overall dimensions of micro or nanoparticles, such as silicon porous particles, may be made already the size of the transmigratory channel and can effectively cross the endothelial cell boundaries.

Plasma membranes from autologous, minimally manipulated ex vivo expanded tumor infiltrating lymphocytes (TIL) (15) can be isolated. TIL can be genetically modified to express a chimeric antigen receptor (CAR) with specificity for CD20 (CD20R) a lineage-specific antigen over-expressed on malignant B cells. By exploiting the potential of TIL to migrate to the tumor microenvironment, TIL plasma membrane containing particle system may be able adhere to the endothelial cell luminal surface undergo transmigration by displaying the LFA-1 protein and may accumulate in the tumor microenvironment through the CD20R tropism.

T-Cell Isolation, Genetic Modification and Ex-Vivo Expansion.

The approach of modifying delivery systems (e.g., microparticles or nanoparticles) with T cell membranes may be used to treat mammalian tumors in both veterinary and human medicine.

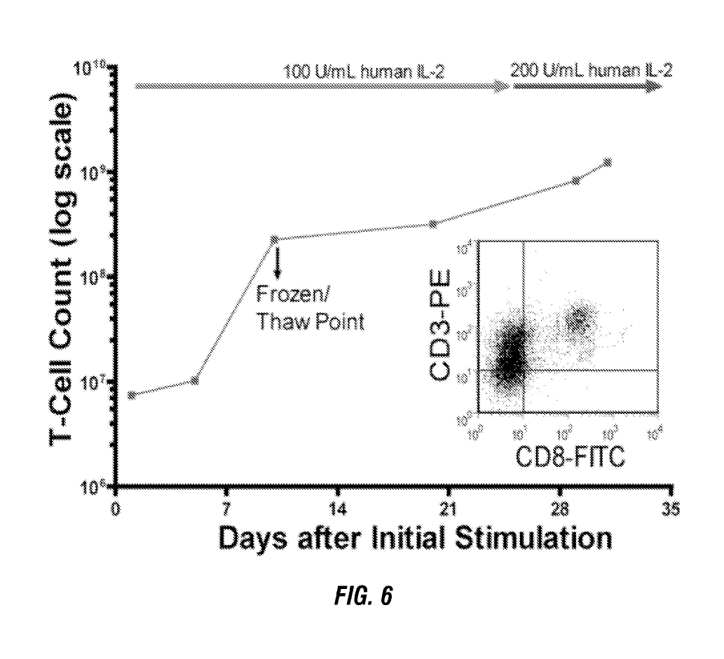

Tumors can be biopsied in order to identify and collect TIL. For example, in humans with melanoma, TIL, when expanded ex vivo, recognize and infiltrate the tumors from which they originated. TIL are numerically expanded on OKT3-loaded artificial antigen presenting cells (aAPC) expressing desired co-stimulatory ligands and the Fc receptor (CD64) for binding exogenous monoclonal antibody (mAb). OKT3 is a mouse CD3-specific mAb that activates human (and other mammalian T-cells, such as, but not limited to canine T cells) for sustained proliferation. When loaded onto K562-aAPC, OKT3 initiates in vitro non-specific activation, proliferation, and cytokine release (FIG. 6).

Large numbers of T cells can be obtained within 14 days (average of 50-fold expansion) generating minimally-manipulated or “young” TIL. These lymphocytes can maintain markers of memory cells. In addition, the lymphocyte populations maintain expression of co-stimulatory receptors (CD27, CD28) and cell surface markers associated with trafficking to the lymph nodes (CCR7, CD62L). The loss of such markers are commonly observed in “older” TILs that were propagated for longer periods of time, or by using alternative approaches. This phenotype can make the young TIL an effective solution for drug delivery because of their ability to potentially traffic to the original tumor sites.

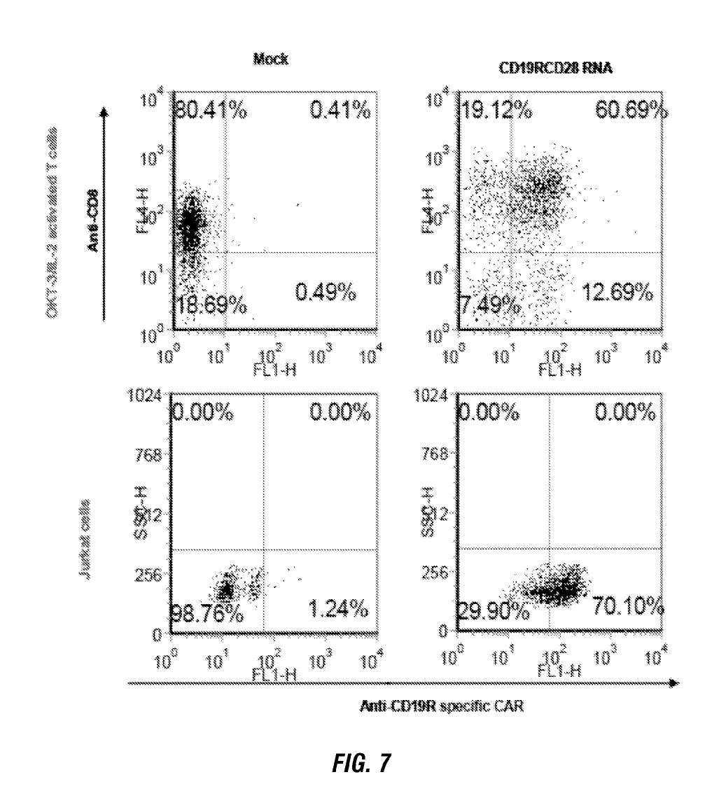

Thus, to improve the therapeutic potential of delivery systems, mammalian T-cells (such as those from humans or dogs) can be rendered tumor-antigen specific in vitro. This can be done by combining immunotherapy with gene therapy by introducing a CAR with specificity for a desired tumor antigen and introducing these receptors using the Sleeping Beauty (SB) transposon/transposase system (see, e.g. Mikkelsen et al. Molecular Therapy (2003), 8, 654-655). The SB system is an advanced non-viral gene transfer strategy with improved safety. The SB system may have cost-benefit over viral vectors. Alternatively, integrating vectors can also express desired CAR transgenes from mRNA that is electro-transferred into ex vivo-propagated T cells. The efficient electro-transfer of in vitro transcribed mRNA has been adapted for clinical use by creating genetically modified CAR⁺ mammalian (human and canine) T-cells (FIG. 7) (16). T cells expressing CD20R can be visualized using a specific fluorescently labeled-antibody against CD20R (15, 16).

Thereafter, the plasma membranes of the modified T-cells may be isolated by methods described previously. Delivery systems of the present invention may then be modified with the isolated plasma membranes.

### Example 2

### Modification of Delivery Systems with Leukocytes

The localization of theranostic particles (NPs) to a tumor site has been the subject of considerable research that so far has not translated into comparably comforting advances in clinical medicine. However, due to limitations in their structure and surface properties, NPs may be unable to overcome the multiplicity of biological barriers (biobarriers) they encounter after intravenous administration. These obstacles may in turn adversely impact NPs' ability to reach the intended target at effective concentrations. The blood-brain barrier, the intestinal lumen endothelium, or the vessel endothelial walls may be prime examples of physical biobarriers to injected agents. The effectiveness of therapeutic and imaging agents may be also hampered by the reticuloendothelial system (RES) that is comprised of macrophages and scavenger endothelial cells that reduce the circulation time and availability of most of the currently developed delivery systems [4, for references in square brackets see REFERENCES LIST 2 BELOW].

Translational research may have improved the ability of theranostic NPs to prolong their circulation time and to reach the target lesion through the use of coating polymers (polyethyleneglycol, PEG) and targeting reagents, such as antibodies, aptamers and recombinant ligands. Considerable advantages may be gained over conventional delivery systems by encapsulating therapeutic drugs in NPs stabilized with a coating that provides targeting capabilities, controlled release and protection from metabolism and degradation. Even though polymer-grafted particles exhibit prolonged residency times in the blood, several studies have indicated that a fraction of intravenously injected long-circulating NPs are rapidly deposited in the liver and the spleen [14, 15, 16].

Since the surface composition of a biomaterial can have an important influence on biologic responses, changing the surface chemistry of a device by coating it with cell components may be a great way to enhance and further improve its circulation properties. The use of biologically-derived coatings may be a fine example of the potential use of biomimetics in the field of surface technologies for biomedical applications. Thus, a hybrid delivery system may be presented as an example of a combination of features and characteristics of natural leukocytes' processes (i.e., tropism towards a tumor site and ability to transmigrate through the endothelial barrier) with nanotechnologies for successfully overcoming biobarriers, while targeting the cancerous lesion.

Rationale

The RES and the endothelial barrier may play a fundamental role in controlling the transport of agents from the blood stream to the surrounding tissues. During an inflammatory event, peripheral blood cells, such as lymphocytes, monocytes and eosinophils, may be recruited through a transendothelial migration (TEM) process. Leukocytes may cross the endothelial wall through paracellular and transcellular routes following a controlled multistep progression that is closely regulated by localized adhesion molecules expressed on the endothelium [31].

Independently of the route taken, leukocytes' TEM may be triggered by the interaction between the intercellular adhesion molecule-1 (ICAM-1) on the endothelial membrane and the lymphocyte function-associated antigen-1 (LFA-1) on the leukocyte membrane. Interaction with ICAM-1 may trigger the activation of endothelial intracellular signaling pathways that may result in extensive cytoskeletal remodeling events that alter endothelial cell contractility and function, facilitating leukocyte diapedesis. During TEM, endothelial cuplike structures enriched in ICAM-1 and LFA-1 surround the site of diapedesis and allow the leukocytes to squeeze through the tight junctions, as they migrate towards the interstitial tumor space [32, 33]. Significantly, TEM does not require any molecular activation in the leukocytes aside from the remodeling of the cytoskeleton to fit the channel that is formed in the endothelial cell. Therefore, TEM is predicted to occur upon contact with leukocyte membrane and does not require an active participation of the leukocytes.

Design

The hybrid system, which may be called a “leukolike” system, may be composed of nanoporous silicon or silica particles (NSPs) preloaded with an active agent, such as an imaging and/or a therapeutic agent. The hybrid system may also compose second stage nanoparticles (NPs), which may contain an active agent. More importantly, the hybrid systems are coated with the cellular membranes of leukocytes freshly isolated from, for example, the peripheral blood.

An imaging agent for imaging diagnosis may be for example, a quantum dot, a near infrared contrast imaging agent, a gold nanoparticle, or an iron oxide particle. A therapeutic agent may be, for example, a chemoterapeutic drug, an antibiotic, a vaccine or a growth factor inhibitor. The load (i.e. the active agent or nanoparticles containing active agent) may be loaded or encapsulated inside the system before the surface coating with the isolated cellular membranes.

The diagnostic and therapeutic agents may be also preloaded into the second stage NPs, such as liposomes or polymer particles, before being loaded inside the system. The system carrying the active agent(s) may be coated or modified with the cellular membranes isolated from cells, such as immune cells.

The surface of the NSPs can be modified with different types of silanes and/or molecular linkers to facilitate the adhesion of the cellular membranes on the different NSPs. NSPs with different roughness and porosity may require different and specific surface functionalization in order to facilitate the interaction of the cellular membranes on their surface.

The hybrid system may present on its surface all the natural components (proteins) that are involved in the biological functions of the leukocytes, including: receptors able to direct the leukocytes towards the cancerous site; and LFA1 (leukocyte function-associated antigen 1), a protein involved in the transendothelial migration from the blood stream to the tumor site.

The composition of the protein exposed on the surface of the “leukolike” system can be opportunely adapted in different ways. For instance, the leukocytes can be ex vivo expanded and genetically modified to express a specific membrane protein. Likewise, one can use liposomes carrying proteins of interest in the lipid bilayer, or in their inner aqueous environment.

Plasma Membrane Isolation and Characterization

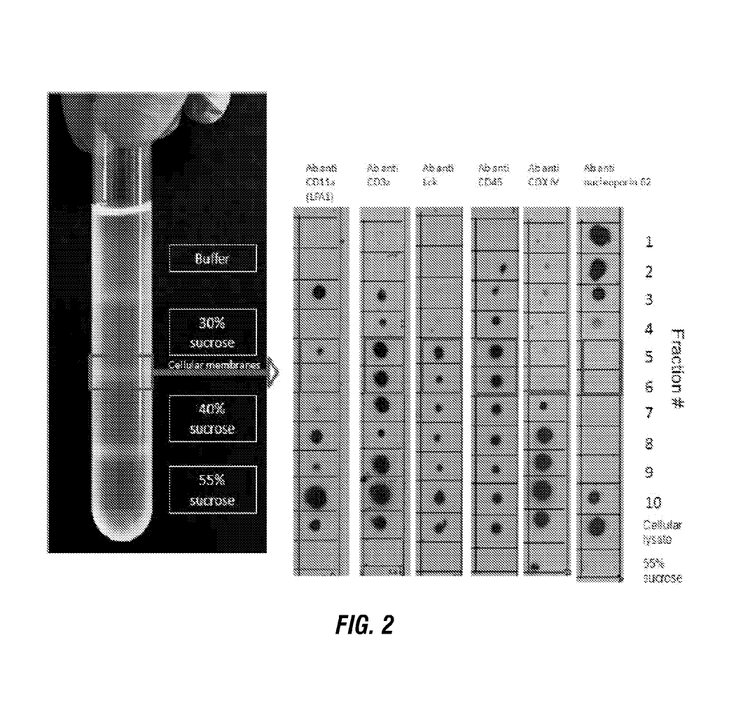

The plasma cellular membranes were isolated from primary leukocytes through a discontinuous density sucrose gradient. Lymphocytes were homogenized in a complete homogenization buffer (HB) by a hand hold dounce homogenizer. The cellular lysate was separated by the cellular debris by centrifugation at a low speed. The supernatants containing the plasma membranes were pooled and laid on a discontinuous sucrose density gradient and ultracentrifuged. After ultracentrifugation, three different lipid white rings were visible along the gradient at the interfaces between the different sucrose layers. In order to identify which one contained the plasma cellular membranes, the distribution of specific protein markers associated with the different kinds of cellular membranes (i.e., nuclear, mitochondrial, and plasma membranes) were used in dot blot of ten fractions collected from the top to the bottom of the gradient (FIG. 2). For nuclear and mitochondrial membranes nucleoporin 62 and COX IV were respectively chosen as markers of interest. To identify plasma membranes, two different markers, Lck and CD45 associated respectively to the lipid rafts and non-lipid rafts membrane regions were tested in order to identify and recover as much plasma membranes as possible. The localization of two additional plasma membrane proteins, CD11a and CD3z, was also verified since these marker proteins are thought to play an important role for the realization of the modified delivery system.

The dot blots results showed that Lck and CD45 were predominantly localized in the fractions numbers 4-6, which correspond to the ring at the 30-40% sucrose interface. Nucleoporine 62 readily localized in the fraction number 3, which corresponds to the ring visible at the supernantant-30% sucrose interface. Likewise, COX IV localized in the fractions 7-8 associated with the lipid ring at the 40%-55% sucrose interface.

While CD3z co-localized with the plasma membrane markers, CD11a co-localized with the nuclear membrane markers. Since CD11a is important for the successive experiments, the fractions containing CD11a were centrifuged at a slow speed in order to remove the nuclear membrane. The supernatant containing CD11a was then added to the fractions associated with the plasma membrane, which also contained CD3z.

The isolated plasma membranes were washed and stored in a normal saline solution at 4° C. In such aqueous solution the hydrophobic interactions among the lipid tails induce plasma membranes to be spontaneously organized into multilayer vesicles with a variable diameter. This multilayer vesicle organization was apparent in the transmission electron microscopy (TEM) images.

Particle Coating/Modification with Plasma Membranes

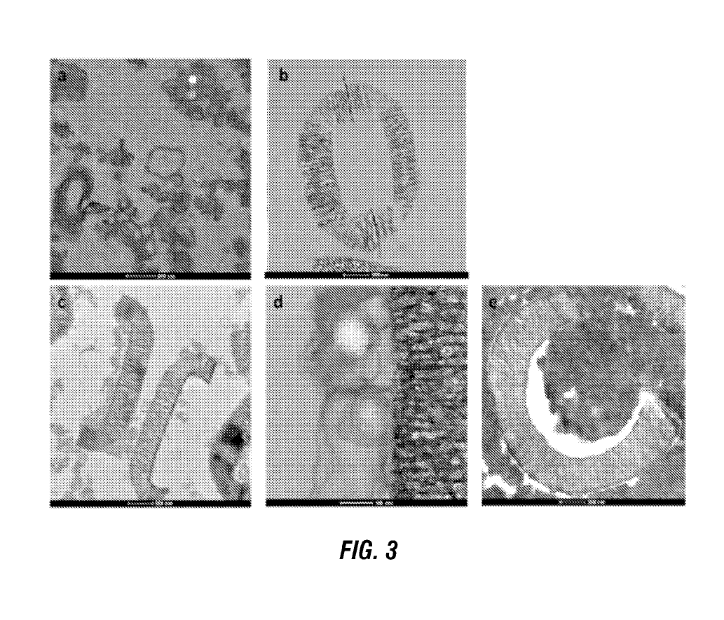

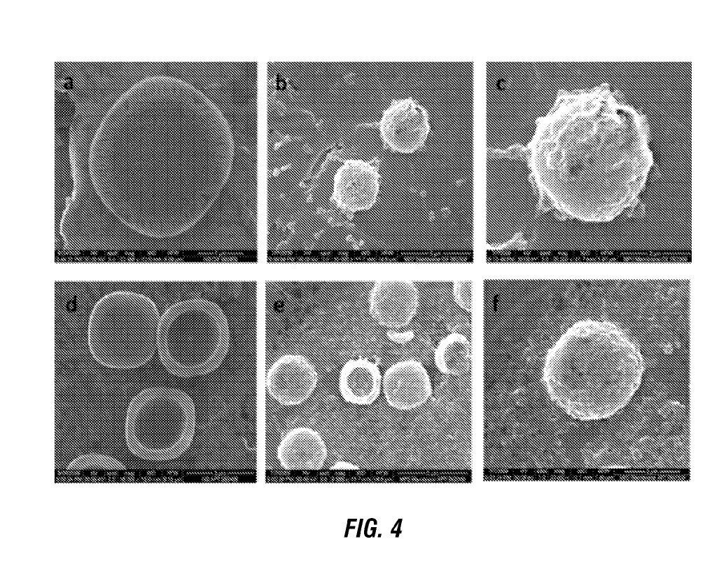

The isolated plasma membranes were incubated with nanoporous silicon particles (NSPs) and/or non-porous particles (silica beads) overnight at 4° C. under continuous rotation. As a consequence of the interaction of the multilayer vesicles and/or liposomes with the surface of the NSPs, the lipid vesicles disintegrated and fused onto the particles' surface. The membrane coated NSPs were visualized by TEM and scanning electron microscopy (SEM) (FIGS. 3 and 4, respectively).

The images show how the cellular membranes adhere around the surface of the particles. Depending on the number of layers in the lipid vesicles, the surface of the silicon particles can be coated with one or more of such lipid layers. The coating ability of these isolated membranes was tested using NSPs and silica beads with and without aminopropyltriethoxysilane (APTES) surface modification. The SEM images (FIG. 4) show that the presence of APTES improved the spreading of the multilayer membrane vesicles all around the particles' surface. This is apparent from the observation that the surface of oxidized NSPs are not as homogeneously coated as the surfaces of the APTES-modified NSPs.

FACS Analysis

The protein composition of the surface of NSPs and silica beads coated with the cellular membranes was characterized by fluorescence activated cell sorting (FACS) analysis. In particular, the focus was on the distribution of plasma membrane proteins CD11a and CD3z. The presence of these proteins can be tested since they can play an important role in the biological function of the modified system.

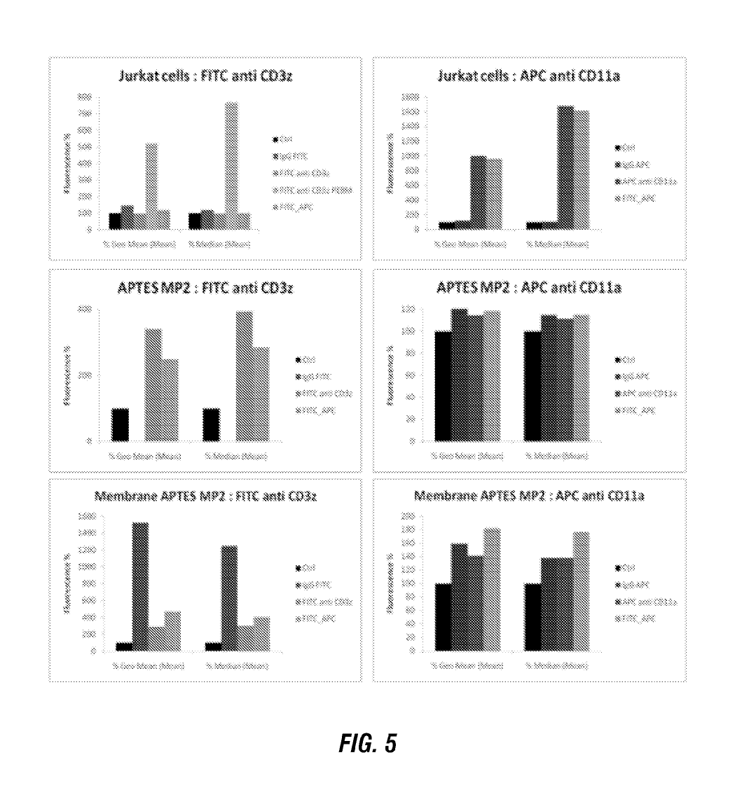

FACS analysis was conducted after staining the membrane coated NSPs with an FITC-conjugated anti-CD3z mAb, and an APC-conjugated anti-CD11a mAb. The results (FIG. 5) show that FITC and APC fluorescence intensity signals increased on the membrane coated NSPs, as compared to the non-coated NSPs used as controls (ctrl).

The FACS analysis can suggests that APTES modified NSPs coated with plasma membranes also register the higher intensity of fluorescence for both the investigated markers. In addition, the results demonstrate that the CD3z associated fluorescent signal is stronger than that associated with the CD11a on the membrane coated NSPs but lower on the leukocyte cells. Without being bound by theory, it is envisioned that the low CD3z expression on the cells is due to the localization of the receptor in the cytoplasmic side of the lymphocyte plasma membranes such that that it becomes inaccessible to the FITC-conjugated anti-CD3z mAb.

After membrane isolation, CD3z remains associated with the membranes. However, CD3z's availability becomes dependent on its localization on the inner or outer part of NSPs surface.

Cell Culture

Primary leukocyte (JurkaT-cell) suspensions were grown in RPMI-1640 medium containing 10% fetal bovine serum (FBS). This medium was supplemented with 1% glutamine. Cells were grown in T-175 ml flasks. The cells were kept in a humidified atmosphere at 37° C. containing 5% CO₂. For the experiments, 2.8×10⁸ cells were used.

Plasma Membrane Isolation

Cells were centrifuged at 500 g for 10 minutes at 4° C. the obtained pellet was re-suspended in 2 mL of HB (0.25 M sucrose, 10 mM Tris/HCl, 1 mM MgCl₂, 1 mM KCl, 2 mM phenylmethylsulfonyl fluoride (PMSF), 200 μg/mL trypsin-chymotrypsin inhibitor, 10 μg/ml DNase, and 10 μg/ml RNase) at pH 7.3. Cells were enucleated in a hand-held Dounce homogenizer (20-30 passes while on ice) and centrifuged at 500 g for 10 minutes at 4° C. The supernatant was then collected and the pellet was re-suspended in HB. The homogenization and centrifugation steps were repeated until the pellet was free of intacT-cells. The presence of intact T-cells in the pellet was verified by light microscopy. The supernatants were then pooled and placed on a discontinuous sucrose density gradient composed of 55% (w/v), 40% (w/v), 30 (w/v) % sucrose in a normal saline solution (NSS, 0.9%).

The discontinuous gradients were ultracentrifuged in a Beckman SW-28 rotor at 20,000 rpm for 30 minutes at 4° C., using polycarbonate tubes. The plasma membrane-rich region was then collected at the 30%/40% interface. Ten fractions were also collected from the top to the bottom of the gradient for successive characterization of the protein distribution along the gradient. The plasma membrane-rich region were diluted two-fold with NSS and ultra-centrifuged in a Beckman SW-28 rotor at 20,000 rpm for 1 hour at 4° C. using polycarbonate tubes. The pellet was then re-suspended in two-fold NSS and ultra-centrifuged in a Beckman SW-28 rotor at 20,000 rpm for 1 hour at 4° C., using polycarbonate tubes. The isolated membranes were then re-suspended in a minimal amount of NSS and stored at 4° C.

Characterization And Protein Distribution Along the Gradient

The distribution of the proteins associated with nuclear, mitochondrial and plasma membranes along the gradient was followed by a dot-blot procedure. For this purpose, 2.5 μl of each fraction were spotted on a polyvinylidene fluoride (PVDF) membrane and immunostained using monoclonal antibodies against nucleoporin p62 (nuclear), COX IV (mitochondrial), Lck (plasma membrane lipid rafts marker), CD45 (plasma membrane non-lipid rafts marker), CD3z and CD11a at 1:5000 dilutions. This was sequentially followed by incubation with an HRP-conjugated mouse anti-human IgG secondary antibody at 1:10000 dilution and enhanced chemiluminescence (ECL) detection.

Preparation of Membrane-Coated Particles

The particles used were nanoporous silicon particles (NSPs) and/or non-porous particles (1.5×10⁶) with diameters of 2.8 μm. The particles were oxidized or superficially modified with aminopropyltriethoxysilane (APTES). The particles were incubated overnight with the washed membranes at 4° C. under continuous rotation. Membranes can also be organized as liposomes by extruding 1 mg of isolated membranes (using a Lipex Biomembranes extruder) 10 times through a 100-nm pore polycarbonate filter (Millipore) under 20 bar nitrogen pressure. The membrane-coated particles were isolated from the un-bound membranes by centrifugation at 500 rpm for 10 minutes at 4° C. The membrane-coated particles were then used for successive analysis.

Sample Preparation for Transmission Electron Microscopy (TEM)

Samples were fixed with a solution containing 3% glutaraldehyde and 2% paraformaldehyde in 0.1 M cacodylate buffer at pH 7.3. After fixation, the samples were washed and treated with 0.1% Millipore-filtered cacodylate buffered tannic acid. The samples were then post-fixed with 1% buffered osmium tetroxide for 1 hour. Next, the samples were stained en bloc with 1% Millipore-filtered uranyl acetate. The samples were then dehydrated in increasing concentrations of ethanol, subsequently infiltrated, and embedded in Spurr's low viscosity medium. Thereafter, the samples were polymerized in a 70° C. oven for 2 days. Ultrathin sections were cut in a Leica Ultracut microtome (Leica, Deerfield, Ill.) stained with uranyl acetate and lead citrate in a Leica EM stainer. The sections were then examined in a JEM 1010 transmission electron microscope (JEOL, USA, Inc., Peabody, Mass.) at an accelerating voltage of 80 kV. Digital images were obtained using AMT Imaging System (Advanced Microscopy Techniques Corp, Danvers, Mass.).

Sample Preparation for Scanning Electron Microscopy (SEM)

The membrane-coated particles are washed three times in millipore-filtered water. Each of the washes was followed by centrifugation at 4500 rpm for 5 minutes at 4° C. 2 μl of each sample was then spotted onto a metal stub and dried inside a vacuum dessicator. Digital images were obtained using a FEI Quanta 400 FEG ESEM equipped with an ETD (SE) detector.

Fluorescence Activated Cell Sorting (FACS) Analysis

The membrane-coated NSPs were stained using a direct staining procedure. Each sample was washed in ice cold 1×PBS containing 10% FBS and 1% sodium azide. The primary labeled monoclonal antibodies (FITC-conjugated anti-CD3z and APC-conjugated anti-CD11a) were then added and incubated for 1 hour at 4° C. in the dark under continuous rotation. After incubation, the samples were washed 3 times by centrifugation at 500 rpm for 10 minutes and re-suspended in the same 1×PBS solution described above. The samples were analyzed on the flow cytometer as soon as possible. FITC- and APC-conjugated isotype matched antibodies were used as negative control.

Confocal Microscopy

Membrane coated NSPs were washed in ice cold 1×PBS, fixed with 1% paraformadehyde, and incubated for 2 hours with the primary anti-CD3z and anti-CD11a monoclonal antibodies that were diluted 1:5000 in 1×PBS containing 1% bovine serum albumin (BSA). Samples were then incubated respectively with secondary goat anti-mouse-IgG Alexa 488 and Alexa 657 monoclonal antibodies for 1 hour and 30 minutes at room temperature in the dark. The samples were then concentrated on a glass slide by a cytospin centrifuge. The fluorescence of the samples were preserved by adding a drop of prolong-gold mounting media. Confocal scanning microscopy of the samples was carried out with a confocal microscopy Leica DM6000 microscope using a 63× oil immersion objective.

20 μg of a green fluorescent lipid (1,2-dioleoyl-sn-glycero-3-phosphoethanolamine-N-carboxyfluorescein) was added to 0.8 mg of lyophilizated isolated cellular membranes and mixed very well. The green fluorescent membranes were incubated with APTES-modified NSPs over night at 4° C. under continuous rotation. The membrane-coated NSPs were then incubated with HUVEC cells in a ratio 5:1. The studies were conducted at three different time points: 2 hours, 4 hours, and 6 hours. At the end of the incubation periods, cells were fixed and permeabilized before the staining with phalloidin Alexa-555 and DRAQ-5 for the visualization of the cytoskeleton and nuclei, respectively.

### Example 3

### Characterization of Cell-like Activity in Leukolike Delivery Systems

Biological barriers still incapacitate therapeutic agents intravenously administered. Although several efforts have been done to improve the stealthiness of drug delivery systems (DDS), they are still unable to efficiently negotiate the phagocytic cells of the immune system and the endothelial cells of the vessels. By taking inspiration from nature, we propose a biomimetic approach for camouflaging DDS among circulating leukocyte cells that, during inflammation are normally recruited at the lesion site via transendothelial migration (TEM). We realized a new generation of DDS, named Leukolike system (LS), by functionalizing the surface of biocompatible nanoporous silicon particles (NSPs) with cellular membranes extracted from leukocytes. By using biological components as natural coating material the LS acquired the same surface composition and functions as those of the donor leukocyte, consequently achieving manifold advantages: a superior evasion of the immune system sequestration, an enhanced TEM while efficiently retaining and release a drug payload.

Introduction

Recent developments in biomaterials open new horizons in biomedical applications especially in the drug delivery field [79-81, for references in square brackets see REFERENCES LIST 3 BELOW]. Although DDS exhibit improved pharmacokinetics and biocompatibility, several efforts are still required to develop new carriers with ameliorate performance properties: prolonged circulation time, site-specific targeting, high loading efficiency, sustained release and reduced side-effects [3, 67]. However, after intravenous injection, these biological actions are strictly affected by several DDS physicochemical factors such as size distribution, shape and surface hydrophobicity that strongly influence the interaction with plasma proteins (opsonins) and the following sequestration by the macrophages of the reticuloendothelial system (RES) [23, 76]. The activation of the immune system together with the endothelial wall represent the major biobarriers that a DDS must overcome to reach the intended target at effective concentrations [33, 82]. The DDS surface functionalization with hydrophilic elements such as polyethylene glycol (PEG) and dextrans significantly increases the blood circulation time by minimizing the opsonins adsorption to their surface [83]. The supplied hydrophilic shell makes them more invisible to the immune system and able to passively extravasate and accumulate at the tumor site via the enhanced permeation and retention (EPR) effect [35, 76].

Based on a self/non-self discriminating mechanism, the immune response triggers an early activation of circulating leukocytes and subsequent recruitment to the lesion site, where they actively contribute to remove the invading agents [38]. The efficiency of the immune system response strictly depends on the rapid shuttling of leukocytes from the bloodstream to the inflammatory site [84]. The leukocyte escape from the vasculature through TEM whether by the paracellular or transcellular routes [57] that involve penetrating manifold barriers: endothelial cells, pericytes and the basement membrane generated by both of these cell types [85]. TEM is predominantly mediated by the interaction between the endothelial intercellular adhesion molecule-1 (ICAM-1) and its counter-receptor lymphocyte function-associate antigen-1 (LFA-1 or CD11a) [86] that triggers the endothelial cellular contractility, required to facilitate the leukocyte diapedesis [87, 88]. However, DDS, currently used, are faraway from reproducing as naturally as possible the structures, components and properties of any blood cell and result unable to completely avoid the recognition by the immune system. Here we realized a LS by camouflaging NSPs [69, 72, 89-91] trough surface coating with cellular membranes extracted from ex-vivo expanded leukocytes. We efficiently transferred both the organic and functional properties of the donor leukocytes to a manmade system, as demonstrated by biochemical and functional analysis. The biological similarity between the leukocyte and the LS surface allowed the LS to escape the macrophage phagocytosis two times more than NSPs. Additionally, in comparison to the NSPs, the LS showed an increased efficiency to retain a payload and maintain the structural integrity by avoiding the lysosomal pathway during transmigration across an endothelial monolayer. While the NSPs mostly stick and release the preloaded drug into the endothelial cells, the LS crosses the endothelial barrier and finally releases the drug in the surrounding tumor cells, accomplishing a tumor cell killing activity twice higher than the NSPs.

The LS, therefore, represents a biomimetic DDS with unique properties that can be adapted to several types of inflammatory pathologies.

Plasma Membrane Isolation and Characterization

By taking advantage from the nature we wanted to recreate the complex properties of the leukocyte cellular membranes on the surface of the NSPs. In order to realize the LS we first attempted to isolate the plasma cellular membranes from both an immortalized line of human T lymphocyte cells (Jurkat cells) and a murine macrophage cell line (J774A.1) by ultracentrifugation in a discontinuous sucrose density gradient.

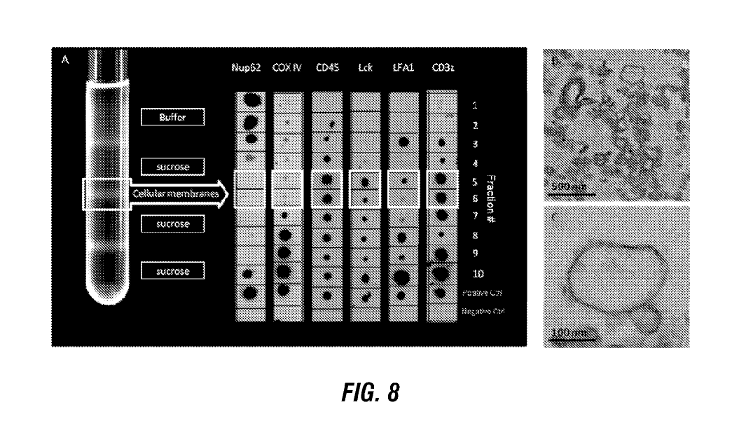

After ultracentrifugation three white lipid rings were observed at the interface between each different sucrose layer. We localized the plasma cellular membranes by screening the distribution of specific proteins associated with the different cellular membranes through immunoblotting on ten fractions collected from the top to the bottom of the gradient (FIG. 8A).

The findings showed that nucleoporin 62 (Nup62) and cytochrome c-oxidase (COX IV), a nuclear and a mitochondrial marker, localized at the supernantant-30% sucrose and at the 40-55% sucrose interfaces respectively; while CD45 and lymphocyte-specific protein tyrosine kinase (Lck), associated with non-lipid raft and lipid raft membrane regions, localized prevalently at the 30-40% sucrose interface. We also studied the localization of the plasma membrane proteins, CD3z and LFA1 (CD11a), essential for the LS realization. CD3z is a component of the T cell receptor (TCR) that participates in the activation of T cells, while LFA1 plays a crucial role in the leukocyte TEM. Since a consistent amount of CD3z and LFA1 colocalized with the mitochondrial and nuclear membranes in the fractions #1-3 and 9-10, we removed the membranes by centrifugation and recovered the supernatants containing CD3z and LFA 1. They were pooled with the fractions containing the CD45 and Lck enriched-membrane (FIG. 8A).

Leukolike System Assembly: Coating of NSPs with Leukocyte Cellular Membranes

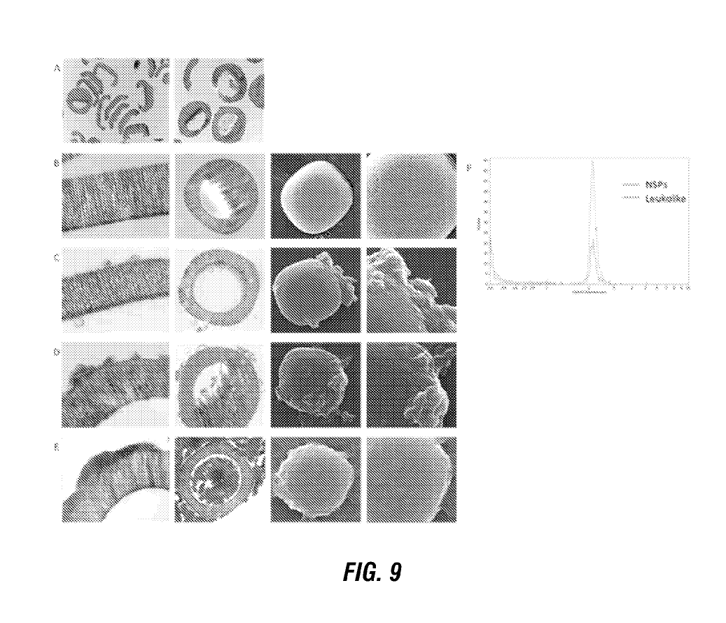

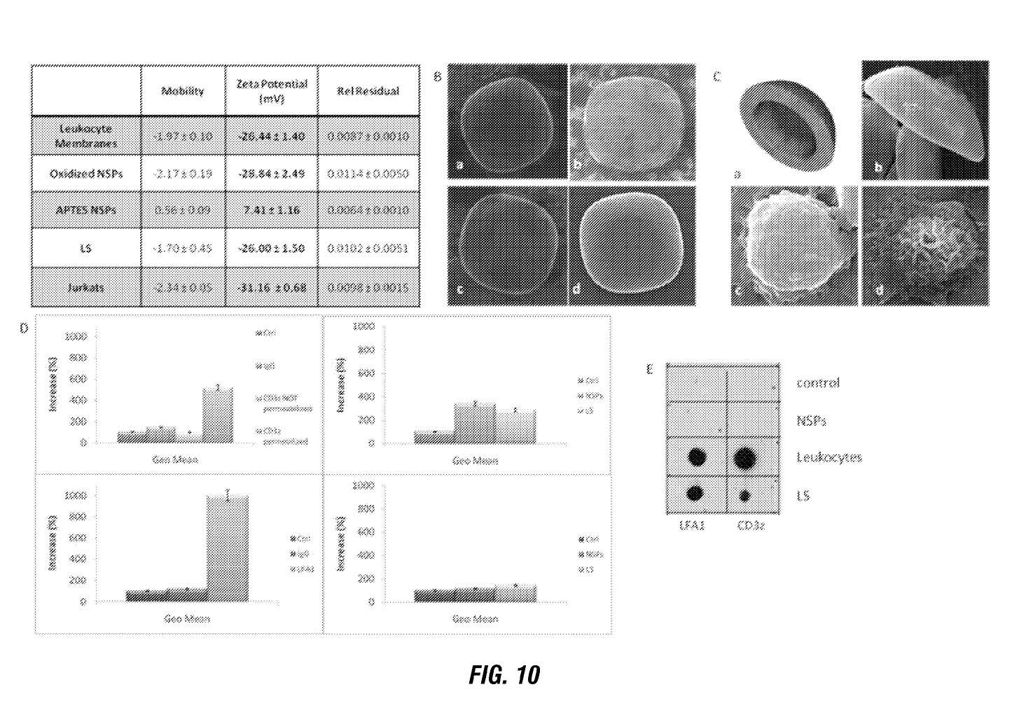

To forge the NSPs surface close to the leukocyte's appearance, we incubated the NSPs with the isolated leukocyte membranes. In an aqueous solution the isolated membranes self-assemble into multi-bilayers lipid vesicles, ranging from the size of 200 nm to 500 nm (FIG. 8B-C), with a net negative surface charge. The surface zeta potentials was measured to be −26.44 mV, similar to the surface charge of NSPs (−28.84 mV) (FIG. 10A). We adopted a direct surface modification approach, based on silanization with aminopropyltriethoxysilane (APTES), to positively charge the NSPs surface (APTES-NSPs), whose zeta potential was measured to be 7.41 mV (FIG. 10A). When the lipid vesicles approached the APTES-NSPs reactive surface, the electrostatic interactions mediate the absorption of the lipid vesicles: once a defect occurs in the outer layer of the lipid vesicles, they fracture and spread on the APTES-NSPs surface. Further spreadings led to the complete coating of the APTES-NSPs by one or more lipid bilayers (FIG. 9C-E). Although multiple lipid bilayers can enclose the APTES-NSPs, the size was essentially unchanged (FIG. 9F).

The relevance of the APTES modification for an optimal surface coating was supported by the non-uniform coating obtained when non-modified NSPs were incubated with the leukocyte membranes (FIG. 10B). However the coating efficiency also depended by the lipid concentration of the coating solution. We prepared two diluted lipid coating solutions (1:2; 1:5) and the APTES-NSPs were partially coated, as predicted (FIG. 9C-E). Additionally, the coating efficiency depended also by the size and lamellarity of the lipid vesicles in the coating solution: smaller vesicles with a reduced lamellarity, obtained through sonication, ensure a more uniform and smooth coating (FIG. 10Cb, Cd). However, the APTES-NSPs coated with membranes organized in larger vesicles better resembled a leukocyte (FIG. 10Cd); hence the name of LS.

Protein Characterization of the LS

We next checked the protein profile of the LS surface by flow cytometry analysis, paying particular attention to the presence of CD3z and LFA1 (FIG. 10D). Under physiological conditions, CD3z is localized in the cytoplasmic leaflet of the membrane bilayer while LFA1 in the extracellular side. Because of their different membrane localizations on the leukocyte cells, LFA1 is detectable at higher levels in comparison to CD3z unless permeabilization of the cellular membrane occurs (FIG. 10D). The lower detection of CD3z and higher detection of LFA1 on the LS rather than on the uncoated NSPs (control) suggested a correct orientation of both the proteins on the LS. However, the lower detection of both proteins on the LS in comparison to the Jurkat cells was justified by the protein loss during the membrane isolation procedure. We also confirmed the presence of CD3z and LFA1 on the LS by immunoblotting (FIG. 10E).

Ability of the LS to Elude the Immune System Response.

The feasibility of transferring some of the physical leukocyte's features to the NSPs and the strong resemblance with a leukocyte led us to verify the LS ability to escape the macrophage uptake trough a self-defense mechanism. We created two different types of LS: the first obtained by coating the NSPs with cellular membranes isolated from Jurkat cells (Jurkat-LS), and the second with membranes extracted from J774A.1 (macrophage-LS).

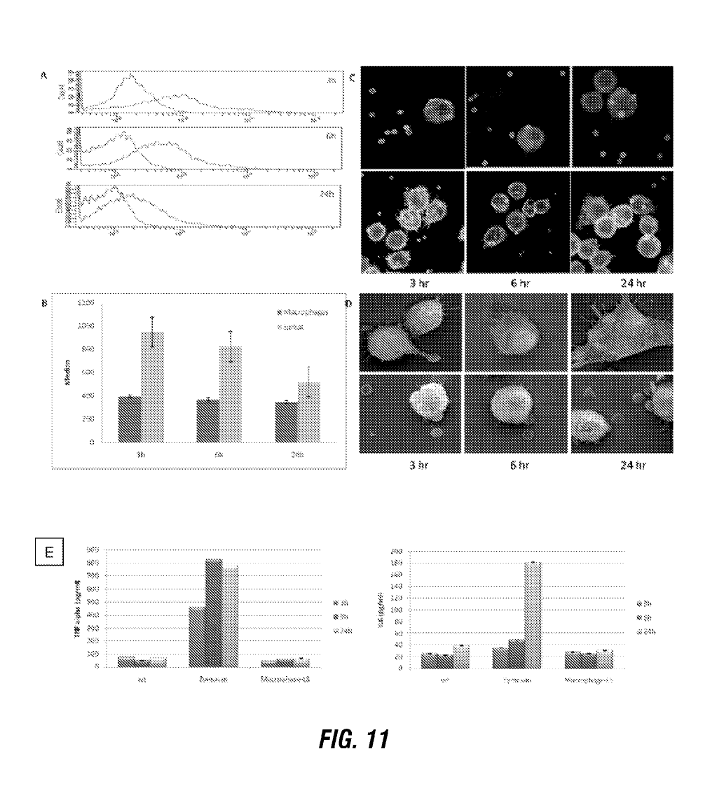

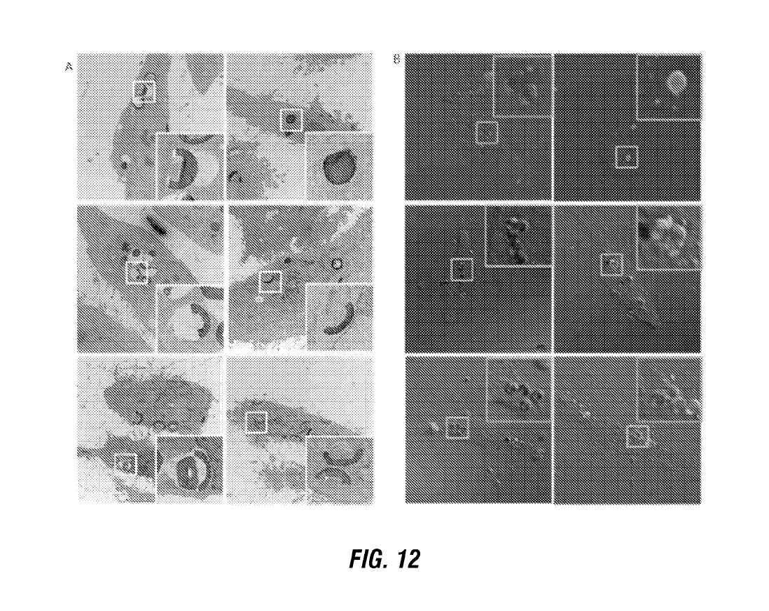

We seeded the J774A.1 at 30% of confluence and incubated with a population of assorted LS (ratio 1:5 cell:LS) for 3, 6 and 24 hr. Although the macrophage's innate tendency to internalize every kind of exogenous agents encountered on their way, the J774A.1 prominently phagocytosed the Jurkat-LS, whereas neglected the macrophage-LS, showing similar surface features (FIG. 11). The results obtained from a flow cytometry internalization assay showed that the uptake rate of the macrophage-LS was constant at each time point and lower than the Jurkat-LS (FIG. 11A-B). The median value observed in presence of macrophage- and Jurkat-LS was 400 and 900 respectively. In particular the highest Jurkat-LS uptake rate (median value 900) was observed after 3 hr of incubation while at 6 and 24 hr it was lower (median values 800 and 500) (FIG. 11B). The lower uptakes observed after 3 hr were probably due to a plateau reached during the macrophage phagocytosis. In order to discriminate the two LS we previously added to the isolated Jurkat and J774A.1 membranes a distinct synthetic fluorescent lipid as a probe. The lipid nature of the probes did not alter the natural composition of the membranes (ratio 98:2 membrane lipids: synthetic lipid). In particular, the Jurkat membranes were labeled with rhodamine-phosphoethanolamine (red fluorescence), while the J774A.1 membranes with a carboxyfluorescein-phosphoethanolamine (green fluorescence).

These results were also confirmed by confocal microscopy (FIG. 11C) and scanning electron microscopy (SEM) (FIG. 11D). At the confocal microscopy J774A.1 were always seen to interact more with the Jurkat-LS and rarely with macrophage-LS, independent of the incubation time. The J774A.1 phagocytic activity was indeed clearly shown in the presence of NSPs, loaded with fluorescein isothiocyanate-bovine serum albumin conjugated to (FITC-BSA) which gives bright green fluorescence. At each time point we observed a ratio between J774A.1 and phagocytosed NSPs of 1:3 (FIG. 11C). Additionally, the SEM micrographs (FIG. 11D) showed that although the macrophage-LS were in close contact with macrophages they remained localized on the cell surface and barely totally engulfed. For the SEM analysis in which the sample needed to be a mixed population of LS we combined together only macrophages-LS and NSPs, due to optical indistiguishibility of Jurkat-LS from macrophage-LS. However all these results univocally confirmed that the J774A.1 did not recognize the macrophage-LS as an exogenous agent as much as they recognized the Jurkat-LS and the NSPs.

We then checked the non-immunogenicity of the LS as a further confirmation of its biocompatibility. We determined the levels of production of two pro-inflammatory cytokines, tumor necrosis factor-alpha (TNF-α) and IL-6, by J774A.1 in response to macrophage-LS (ratio J774A.1:macrophage-LS 1:5) after 3, 6 and 24 hr of interaction. A zymosan solution (1 ng/ml) was used as positive control. We observed that zymosan induced high levels of TNF-α (450 pg/ml) immediately after 3 hr, and of IL-6 (180 pg/ml), only after 24 hr; while the macrophage-LS induce the secretion of basal levels of both TNF-α (<100 pg/ml) and IL-6 (<40 pg/ml) (FIG. 11E), as well as macrophages. We also showed the non-immunogenicity of NSPs and Jurkat-LS (data not shown).

Interaction of the LS with HUVEC

We investigated the LS behavior in a cellular environment by using a well established endothelial model of large vessel endothelium (HUVECs). For these experiments we seeded HUVECs at 70% confluence and treated for 24 hr with TNF-α, in order to induce an inflammatory response. After TNF-α stimulation, the media was removed and replaced with the experimental media containing the NSPs and the LS at a ratio HUVECs:NSPs/LS 1:5. The subcellular localization within the endothelial cells was detected after 3, 6 and 24 hr of incubation by transmission electron microscopy (TEM). We observed that at each time point, the LS were always surrounded by the cellular cytoplasm and maintained their integrity showing patches of coating membranes still adherent to the surface, whereas the NSPs (control) were localized into intracellular vesicles (FIG. 12A) well defined by a lipid bilayer. Since cells usually phagocytate DDS into phagosomes, intracellular vesicles that mature in phagolysosomes by fusing with lysosome vesicles [92], we analyzed their subcellular localization with lysosomal apparatus using the RED Lysotracker lysosomal staining. The endothelial cells were treated with the RED Lysotracker solution (10 ng/ml) for 1 hr by the end of each time point and immediately observed, in live, at the confocal microscope. The cells incubated with the NSPs showed a clear colocalization with lysosomes (red spots) already after 3 hr. On the other side, cells incubated with the LS (green signal associated to the coating membranes) showed no colocalization at any of all the three time points (FIG. 12B), confirming the previous TEM results. These observations indicated that the presence of the coating membranes altered the phagocytosis pathway by inducing the lysosomal escape, as leukocytes.

Intracellular Retainment of the LS Payload

Having observed the integrity of the LS after cellular internalization, we tested the efficiency of the LS to retain and delay the release of a payload within a cell, in comparison to the NSPs. For this purpose we preloaded NSPs with doxorubicin (DOX) before the leukocyte membranes coating. For an homogeneous coating it was required to maintain the system in continuous movement, thus causing the early release of some loaded DOX. As a consequence, the amount of loaded DOX was considered the final amount (0.061 mg) left after the coating. The same treatment was applied to the non-coated NSPs (0.059 mg).

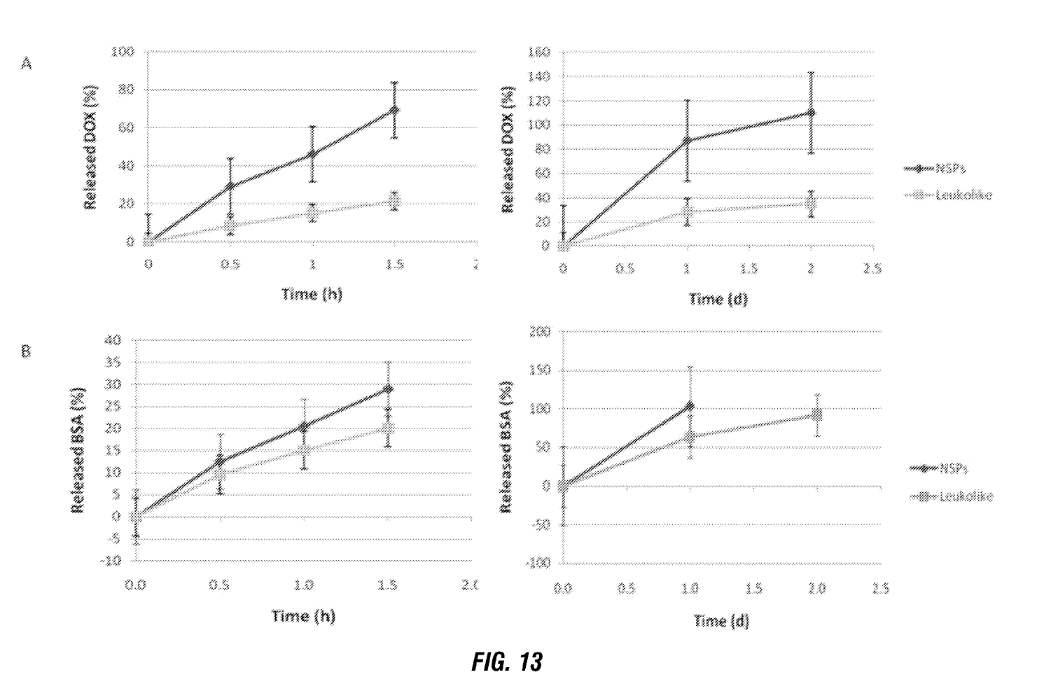

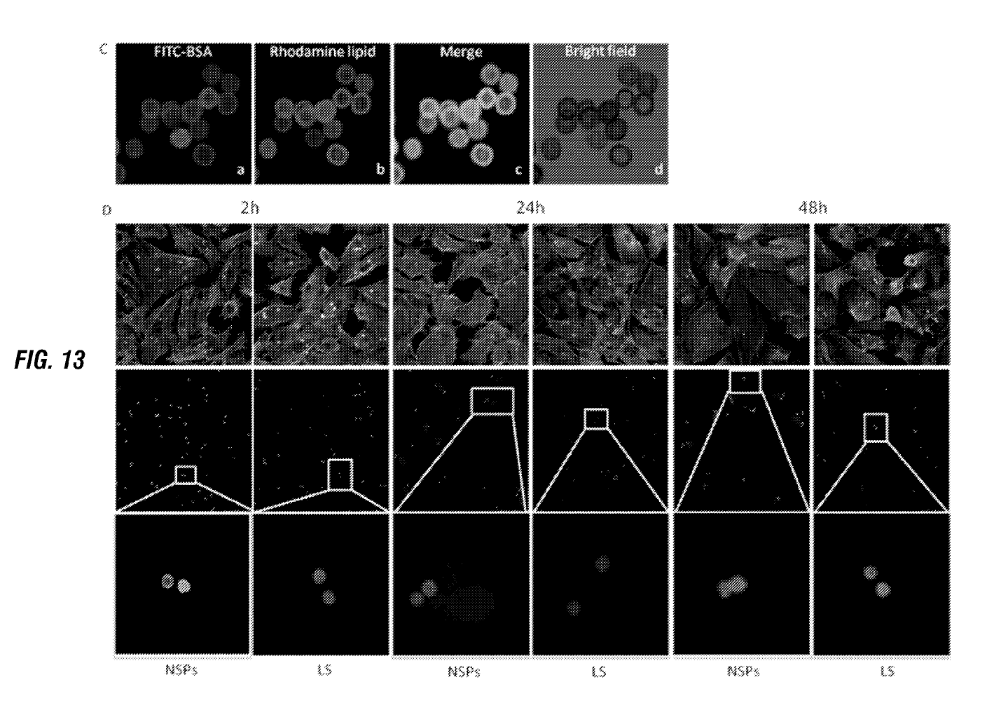

The release study was conducted leaving both the NSPs and the LS at 37° C., in continuous movement. We checked the release after 30 min, 1 and 1.5 hr (burst release) as well as after 1, 2 and 3 days (sustained release) (FIG. 13A). At each time point the samples were centrifuged at 500 rpm, the surnatants were saved and the pellets resuspended into 250 μl of fresh phosphate buffered saline (PBS), pH 7.2. The drug concentration was estimated as a linear function of the absorbance, read at 480 nm, as determined by the standard curve. The DOX burst release from the two systems was really different: after 1.5 hr, 80% of loaded DOX was already released from the NSPs, while only the 20% from the LS. Consequently, after 2 days, when the NSPs totally released the loaded DOX, the LS released only the 40% of the payload, suggesting a retaining function of the coating membranes (FIG. 13A). The release profile did not change by changing the loaded agent. We repeated the experiments using FITC-BSA as payload (FIG. 13B). In this case a prolonged retainment of FITC-BSA from the LS was observed until the second day while after 1 day the NSPs released 100% of the loaded FITC-BSA (FIG. 13B).

On the basis of these results, the LS retaining property was also checked in a cellular system. We incubated the NSPs (control) and the LS, both carrying FITC-BSA (FIG. 13C), with TNF-α activated HUVECs (70% confluence), in a HUVECs:NSPs/LS ratio of 1:5. The coating membranes were labeled with the synthetic red fluorescent lipid for tracking their intracellular fate. The release was checked after 2 hr, 1 and 2 days looking for the FITC-BSA fluorescent signal at the confocal microscope. The expected green fluorescence intensity in the area surrounding the particles was observed only after 24 hr within the cells carrying the NSPs. At the same time point no significant green fluorescent signal was observed around the LS, even thought a diffuse red fluorescence intensity was observed into the cytoplasm. After 48 hr the green fluorescent signal was stronger and more spread all around the NSPs. An increased green fluorescence intensity was also seen in the area surrounding the LS, where the red fluorescent signal became stronger too (FIG. 13D). The observed results confirmed the theory of the preserved integrity of the LS for 24 hr that justifies the delayed release of the payload, both within and without the cells, by comparison with the NSPs.

Transmigration Ability of the LS

Thinking at the LS as a device able to mimic the leukocyte properties, we verified its ability to transmigrate through the endothelial monolayer, like real leukocytes, while delivering a therapeutic agent.

We examined the transmigration ability using 24-well transwell inserts constituted by a polycarbonate microporous membrane with a 8 μm pore size. We seeded HUVECs (about 4×10̂5) on the upper side of the transwell membrane and let them adhere and spread until 100% confluence. We established the best condition to obtain a confluent HUVECs monolayer after seeding HUVECs at different dilutions and checking the confluence with crystal violet staining When the monolayer reached 100% confluence, the media in the upper chamber was replaced with experimental media containing DOX loaded NSPs or LS in a concentration that respected the ratio 1:5 cell:particles. The cells were incubated for 24 hr allowing the particles to transmigrate toward the underside of the transwell insert. The number of NSPs and LS that migrated to the bottom surface of the wells was determined by acquiring at the optical/fluorescent microscope four non-overlapping random fields on each well. The experiments were repeated in triplicate. The particle number of each field was estimated using ImageJ software and the averages reported in the graph (data not shown).

The results showed that the transmigration ability of the LS was higher than the NSPs one. As a following step we tested the tumor killing ability of the LS on breast cancer cells (MDA-MD-231) seeded at the bottom of the lower chamber of the transwell system. After crossing the endothelial monolayer the LS interact with the MDA-MD-231 cells while releasing the DOX payload (0.39 mg). The DOX-associated cytotoxic effect was evaluated by MTT assay and we observed that the cell viability decreased rapidly in presence of LS than NSPs as a dose-dependent result. MDA-MD-231 treated with and without free-DOX were used as controls (data not shown).

### DISCUSSION

In biomedical research the most important concern raised related to the way of controlling the physical-chemical properties of materials for obtaining a specific biological behavior [93]. In order to achieve this aim, researchers started to investigate the surface features of the body's own cells, such as the blood cells.

We proposed the LS as a feasible example of a synergic combination between artificiality and nature. To our knowledge the LS represents the first successful attempt to realize an innovative DDS that integrates the features of an artificial delivery system (biodegradability, biocompatibility, agent loading and release) with the natural properties of the leukocytes (free circulation in the blood stream, TEM and tropism towards the inflammatory site). By combining together all these properties we aimed to create a hybrid system able to reach the intended site successfully and to release a theranostic agent at an effective concentration.

So far we demonstrated that the leukocyte cellular membranes can be used as a natural coating material, ideal to confer on artificial device some of the properties of a blood cell. A part from showing the same protein/lipid composition, the LS also resembled the figure of a leukocyte, minimizing its recognition from the immune system. By evading from the macrophage uptake, the circulation time increases, offering to the LS a higher chance to reach the interested site. The LS thus looks more promising than others DDS that, on equal circulation time, require different chemical surface modifications that make them more visible to the immune system.

Moreover our hybrid system showed the ability to overcome the endothelial cells lining a vessel wall. During the TEM, the LS escapes the lysosome pathway, at which any exogenous agent is commonly destined, preventing the enzymatic degradation of the coating membranes and a burst release of DOX at the endothelial level. We believed that the intracellular interaction between LFA1, exposed on the LS surface, and ICAM1, expressed in the stimulated HUVECs, activated the signaling pathways involved in the endothelial cytoskeletal remodeling and contractility during the leukocyte diapedesis [94]. The active interaction between the proteins on the LS surface and the endothelial cells during the transmigration was confirmed by the lower transmigration rate of the NSPs. Probably NSPs could transmigrate only through the paracellular route and not by the intracellular pathway that is mediated by LFA1/ICAM1 interaction. Additionally, the NSPs entrapped into the lysosomal vesicles were probably degraded or eventually released very late.

The LS thus showed some of a leukocyte properties increasing the possibility to reach the inflammatory site and release the payload in response to the environmental conditions. The acidic pH of the tumor matrix and the secreted enzymes will lead to the coating membrane dissolution and the consequent DOX leaking and uptake by tumor cells. The LS can be also internalized by the tumor cells and release the therapeutic agent directly into their cytoplasm.

In conclusion, all these properties suggested a possible biomedical application of the LS as optimal DDS. In the cancer therapy, however, a specific targeting strategy is required. Before feature testing on animal models, the targeting ability of the LS will be improved by using cellular membranes of primary leukocytes ex-vivo expanded and genetically modified. The possibility to improve the leukocyte tumor tropism by genetically inducing the expression of specific tumor targeting agents offers an additional tool to optimize the LS and to apply to different cancer types as well as to all the vast array of pathologies which involve inflammation.

Future Perspective

Several types of DDS have demonstrated to improve the therapeutic index of the carried drug while reducing their side effects, after intravenous administration. Although currently available DDS can enhance the drug accumulation at the interested site, especially in tumors, the DDS interactions with and uptake by the tumor cells remain insufficient. The main strategy that was proposed to further enhance drug delivery and retention at the level of tumor cells is based on the active targeting [95], that guaranties a highly specific biodistribution of the carrier due to specific interactions.

One way to promote recognition between DDS and target cells is to attach ligands at the DDS surface that can bind specifically to target cells. For instance, proteins or carbohydrates can be used as ligands of endogenous receptors expressed at the cell surface [96, 97]. An advantage of using specific ligands as targeting moieties is that the DDS would be targeted only toward cells showing high expression levels of receptors in comparison to physiological conditions. In fact, it is common in several cancer cells to observe an over-expression of some receptors, whereas other are downregulated and almost disappear from the cell surface. Thus, it seems useful to take advantage of differences in the level of receptor expression in the targeting strategy.

However, the actual methods for grafting various types of targeting moiety on DDS surface have to be improved in order to preserve the functionality of the ligand active site. The ideal DDS should be able to specifically target the cancer lesion while still maintaining the ability to escape the immune system and overcome all the other biological barriers.

Towards a Clinical Application of the Leukolike System

According to these requests, a new efficient method for grafting targeting moieties can derive from the integration of biotechnology with immune-based and gene therapy-based approaches, which can have wide applications across the field of drug delivery.

Additionally, the combination of technology with nature in order to adapt the physiological mechanisms adopted by the immune cells to NSPs, already brought to the development of the LS a new class of DDS able to avoid unwanted uptake and clearance from RES and thereby to improve the circulation time of the first stage vectors (NSPs).

As proposed in the chapter above, the surface functionalization of NSPs with leukocyte plasma membranes can be used as a “stealth approach” to provide a physical protection against RES uptake and an increased accumulation at the tumor level with a consequent prolonged cytotoxic activity. The coating membranes, indeed, prevent the burst release of the payload thus ensuring a prolonged activity of the drug in the time.

Thinking to an imminent test of these successful results in an in vivo model, eventually, it will be interesting to actively target the LS by transferring on it the natural tropism that circulating leukocytes have for the tumor site. If proved favorably, consequently, this technology will be translated to a pre-clinical model in which autologous tumor infiltrating lymphocytes (TIL) can be directly collected by biopsy of tumor samples and ex vivo expanded.

It is also known that after ex vivo expansion TILs are still able to recognize and infiltrate the tumors from which they originated [98, 99]. Moreover ex vivo expanded TIL still maintain markers of memory cells, co-stimulatory receptors and cell surface markers associated with their trafficking to the tumor [100]. The TIL can also be genetically modified in order to express high levels of a chimeric antigen receptor (CAR) with a particular specificity for a lineage-specific antigen of interest, that is known to be over-expressed on tumor cells.

This kind of gene-therapy approach improves the tumor-antigen phenotype of the young TIL making them and consequently the LS, an attractive solution for drug delivery because of their increased ability to specifically recognize the tumor cells.

The plasma membranes of the TIL that efficiently express CAR will be isolated and used for the realization of LS of a second generation.

By exploiting the potential of autologous TIL to migrate to the tumor microenvironment, it can be expected that the LS system, upon intravenous injection, will move toward the luminal surface of the endothelial cell drizzling the tumor mass, where will undergo transmigration and finally accumulate in the tumor microenvironment due to the CAR tropism.

In this way, by perfectly combining the rational design of a DDS with the fundamental understanding of tumor biology, that is a necessary step to better overcome the numerous barriers encountered, the LS represents the ideal approach to determine a successful and personalized case-by-case strategy. The LS approach thus offers the possibility to create, time by time, LS with different properties able to translate a laboratory-based research into a real therapy, better suited to face the tumor heterogeneity.

The versatility of the LS, due to the natural properties of its components (autologous leukocytic membranes) and to the ability to genetically modify primary leukocytes with the desired tumor targeting agents, offers a powerful tool applicable not only to a multitude of different cancer types but to inflammatory pathologies in general.

Material and Methods

Cell Cultures.

The immortalized T lymphocytes cell line (Jurkat), the murine macrophage cell line (J774A.1), the human umbilical vein endothelial cell line (HUVEC) and the human breast cancer cell line (MDA-MB-231) were all purchased from the American Type Cell Collection (ATCC). Jurkat cell suspensions were grown in RPMI-1640 medium (GIBCO) supplemented with 10% fetal bovine serum (FBS), 1% glutamine and 1% antibiotic antimycotic solution (Pen-Strep). J774A.1 and MDA-MB-231 cells were cultured in a-minimum essential medium (a-MEM) supplemented with 10% FBS and 1% Pen-Strep. HUVEC were cultured in recommended EGM-2-MV medium supplemented with EGM-2-MV singlequots and 5% FBS. The cells were kept in a humidified atmosphere, at 37° C., containing 5% CO₂.

Plasma Membrane Isolation

2.8×10⁸ cells were centrifuged at 500 g for 10 min at 4° C. and the pellet resuspended in 2 mL of complete homogenization buffer (HB) (25 mM sucrose, 10 mM Tris/HCl, 1 mM MgCl₂, 1 mM KCl, 2 mM phenylmethylsulfonyl fluoride (PMSF), trypsin-chymotrypsin inhibitor 200 ug/mL, DNase 10 ug/ml, RNase 10 ug/ml final concentration; Sigma-Aldrich) pH7.3. Cells were enucleated in a hand-held Dounce homogenizer (20-30 passes in ice) and centrifuged at 500 g for 10 min at 4° C. The supernatant was collected and the pellet resuspended in HB. The homogenization and centrifugation steps were repeated until the pellet was free of intact cells, checked by light microscopy. The supernatants were pooled and lied on a discontinuous sucrose density gradient composed of 55% (w/v), 40% (w/v), 30% (w/v) sucrose in a 0.9% normal saline solution (NSS). The discontinuous gradients were ultracentrifuged in a Beckman SW-28 rotor at 28.000 g for 30 min at 4° C., using polycarbonate tubes. The plasma membrane-rich region was collected at the 30/40% interface. Ten fractions were collected from the top to the bottom of the gradient for successive protein characterizations. The plasma membrane-rich region was diluted two-fold with NSS and ultracentrifuged in a Beckman SW-28 rotor at 28.000 g for 1 h at 4° C. The pellet was resuspended in two-fold NSS and ultracentrifuged again at the same conditions. The isolated membranes were lyophilized over night, weighted and stored at 4° C. after rehydratation in a minimal amount of NSS.

Immunoblotting

The distribution along the gradient of proteins associated to nuclear, mitochondrial and plasma membranes was analyzed by dot-blot procedure. Briefly, 2.5 μl of each fraction were spotted on a polyvinylidene fluoride (PVDF) membrane. The membrane was blocked with 5% milk, 0.1% Tween-20 in PBS solution, followed by sequential incubation with primary antibody (1:5000 dilution) and HRP-conjugated mouse anti-human IgG secondary antibody (1:10000 dilution) (SantCruz Biotechnology). The blots were developed using SuperSignal West Dura chemiluminescent substrate (Pierce) and the luminescent signals recorded on X-ray film using a Konica SRX-101A X-ray processor. The monoclonal antibodies (mAb) used as primary Ab were: anti nucleoporin p62 (np62), COX IV, Lck, CD45, CD3z (SantaCruz Biotecnology) and LFA1 (CD11a) (Biolegend).

LS Assembly

The protein concentration of the isolated plasma membranes was quantified by Bradford assay (BioRad). The lipid concentration was estimated considering the protein to lipid ratio 1:1 by weight. The membrane solutions were always diluted in a such way to have a final lipid concentration of 1 mg/ml.

NSPs (1.5×10⁶) with a diameter of 2.8 μm, oxidized or superficially modified with APTES, were incubated with the lipid membrane solution over night, at 4° C. under continuous rotation. In some conditions the membrane solution was sonicated for 45 min at 45° C. before incubation with the NSPs. Lipid membrane solutions with a dilution factor of 1:2 and 1:5 were also prepared.

After incubation, the not-bonded membranes were washed away from the membrane coated NSPs (LS) by centrifugation at 500 rpm for 10 min at 4° C. The same conditions were applied for all the LS realized (Jurkat-LS and macrophage-LS were realized using membranes isolated from Jurkat and J774A.1 cells respectively).

Fluorescent LS were realized for flow cytometry and confocal microscope analysis. A synthetic fluorescent lipid (1,2-dioleoyl-sn-glycero-3-phosphoethanolamine-N-carboxyfluorescein (PE-FITC) or 1,2-dioleoyl-sn-glycero-3-phosphoethanolamine-N-lissamine-rhodamine-B-sulfonyl (PE-Rhod)) was resuspended into the lipid membrane solution, with a final lipid molar ratio 2:98. The molecular weight of the phosphatidylcholine was considered as the mean molecular weight value of the isolated membranes.

The ζ-potential measurements of NSPs, LS, lipid membrane solutions and Jurkat cells were performed in phosphate buffer (pH 7) using a Zeta PALS Zeta Potential Analyzer (Brookhaven Instruments Corporation; Holtsville, N.Y.). The average sizes were determined at the Multisizer™ 4 Coulter Counter (Beckman Coulter).

Transmission Electron Microscopy (TEM)

The samples were fixed with a solution containing 3% glutaraldehyde (GTA) and 2% paraformaldehyde (PFA) in 0.1 M cacodylate buffer, pH 7.3. After fixation, the samples were washed and treated with 0.1% Millipore-filtered cacodylate buffered tannic acid, postfixed with 1% buffered osmium tetroxide for 1 h, and stained en bloc with 1% Millipore-filtered uranyl acetate. The samples were dehydrated in increasing concentrations of ethanol, infiltrated, and embedded in Spurr's low viscosity medium. The samples were polymerized in a 70° C. oven for 2 days. Ultrathin sections were cut in a Leica Ultracut microtome (Leica, Deerfield, Ill.) stained with uranyl acetate and lead citrate in a Leica EM stainer, and examined in a JEM 1010 transmission electron microscope (JEOL, USA, Inc., Peabody, Mass.) at an accelerating voltage of 80 kV. Digital images were obtained using AMT Imaging System (Advanced Microscopy Techniques Corp, Danvers, Mass.).

Scanning Electron Microscopy (SEM)

Samples were spotted onto a metal stub, dried inside a vacuum desiccators over night and coated with a thin layer of gold (5 nm) using an SEM gold sputter before the acquisition of digital images using a FEI quanta 400 ESEM FEG instrument equipped with an ETD (SE) detector or a Hitachi S-5500 SEM apparatus.

Flow Cytometry

Surface staining of Jurkat cells (1×10̂6), NSPs (2×10̂5) and LS (2×10̂5) were performed in ice cold PBS 1×, 10% FBS, 1% sodium azide. Primary labeled antibodies, FITC-conjugated anti-CD3z mAb or/and APC-conjugated anti-CD11a mAb, were added and incubated for 1 h at 4° C. in the dark under continuous rotation. For the intracellular staining of the CD3z domain, Jurkat cells were previously permeabilized in 0.01% Tween-20 for 4 min Opportune isotypes of the IgG FITC- and APC-conjugated mAb were used as negative control at the same conditions. After washing, the samples were then analyzed with a Becton Dickinson FACS Calibur equipped with a CellQuest software. Five thousand events were evaluated for each experiment. The results are the average of three experiments.

Macrophage Uptake of the LS

J774A.1 cells were seeded at 30% confluence and incubated with: APTES-NSPs alone (control), Jurkat-LS alone (positive control), macrophage-LS alone (negative control), a mixed population containing NSPs and macrophage-LS (for SEM analysis), Jurkat- and macrophage-LS labeled with PE-Rhod and PE-FITC respectively (for confocal microscopy and flow cytometry); with a ratio cell:particles 1:5. The samples were analyzed after 3, 6 and 24 hr incubation by SEM, confocal microscopy and flow cytometry.

For SEM analysis samples were fixed using a solution containing 2.5% GTA. After fixation, the samples were washed and dehydrated using 30, 50, 70, 90, 95 and 100% ethanol serial dilution steps, followed by dehydratation in 50% ethanol-hexamethyldisilazane (HMDS) and pure HMDS solution. Samples were dried for 2 days in a desiccator before sputter coating with 5 nm layer of gold and observation by using a FEI quanta 400 ESEM FEG instrument.

For the confocal microscopy analysis, the J774A.1 cells, seeded in 4 chambers glass slides, were fixed in 4% PFA solution for 20 min, washed two times with PBS, permeabilized using 0.1% Triton-X 100 solution for 4 min and washed with PBS two times. After 30 min incubation with 1% bovine serum albumin (BSA) blocking solution, the cellular cytoskeleton staining was performed with Alexa-Fluor 594-Phalloidin (Invitrogen) for 30 min, followed by the nuclear staining with DRAQ5 (Biostatus Ltd) for 45 min. All the steps were completed at room temperature (RT), by preventing light exposure. After staining, the chambers were removed, a drop of ProLong Gold mounting medium was added and the coverslip mounted on. The samples were observed using a Leica DM6000 upright confocal microscope equipped with a 63× oil-immersion objective.

A flow cytometry analysis was accomplished to quantify the percentage of PE-FITC and PE-Rhod positive cells as a measure of macrophage uptake. The J774 cells were detached from the wells by gentle scraping with a cell scraper, fixed with 4% PFA and analyzed with a Becton Dickinson FACS Calibur equipped with a 488-nm Argon laser and CellQuest software.

Cytokines Analysis

J774A.1 macrophages were cultured overnight in 24-well plates at a 30% confluence containing 1 mL medium. After 24 hr the cells were incubated with fresh medium (600 μl) containing NSPs, macrophage-LS, Jurkat-LS and macrophage-/Jurkat-LS (1:5 cell:particles). Zymosan at 10 ng/mL concentration (Sigma, USA) was used as a positive control for cytokines production and untreated cells were used as a measure of basal levels of cytokine release. The cell culture supernatant was collected at 3, 6, and 24 hr and stored at −80° C. Samples were analyzed according to the manufacturer's instructions using a Abcam mouse-TNF-α and mouse IL6 cytokine kit ELISA (Abcam). Cytokine levels were read on a SPECTRA max M2 plate reader (Molecular Devices). The quantification was carried out based on standard curves for each cytokine in the concentration range of 1-1000 and 1-500 pg/ml respectively.

LS Interaction with Endothelial Cells and Subcellular Localization

HUVECs were grown until 70% confluence and stimulated with tumor necrosis factor alpha (TNF-α) 10 ng/mL, for 4 h at 37° C. After activation HUVECs were incubated with APTES-NSPs (control) and LS labeled with PE-FITC (ratio cell:particles 1:5) for 3, 6 and 24 h at 37° C. Samples were prepared for TEM analysis as previously described. In some experiments cells were incubated for 1 h by the end of the incubation time with a Red LysoTracker solution 10 ng/ml for the lysosomal staining. The LysoTracker solution was washed away with PBS-glucose buffer (GIBCO). Live images were acquired within 1 h using a Leica DM6000 upright confocal microscope equipped with a 63× oil-immersion objective.

LS Loading and Release Profile of a Payload

APTES-NSPs (1×10⁸, 2.8 μm) were resuspended into 200 μl of a FITC-BSA solution (5 mg/ml) for 2 h at 4° C. in the dark, under continuous rotation. Samples were then centrifuged at 2000 rpm (Beckman Coulter Allegra X-22 Centrifuge equipped with a 296/06 rotor) for 5 min to remove the free unloaded FITC-BSA. The amount of FITC-BSA in the supernatant was quantified evaluating its emission peak at 488 nm using a UV-vis spectrophotometer. The fluorescence was converted into a concentration (μg/mL) of BSA using a standard curves obtained at known FITC-BSA concentrations. The FITC-BSA loaded NSPs were mixed with 200 μL of 1 mg/ml coating membrane solution and incubated at 4° C. for 2 h under continuous rotation. FITC-BSA loaded NSPs used as control were subjected to the same procedure. Samples were then centrifuged at 1000 rpm for 5 min to remove the no-bonded membranes and the amount of FIT-CBSA released during the coating step. The amount of loaded FITC-BSA left into the NSPs and the LS was then estimated.

The release profile of FITC-BSA from NSPs and LS was evaluated maintaining the systems in a moving condition. The supernatant was taken out at established time (30 min, 1 hr, 1.5 hr, 24 hr, 48 hr, 72 hr) and replaced with 200 μL fresh NSS. The fluorescence of FITC-BSA at 488 nm was reported and the cumulative release of FITC-BSA was calculated. Statistical analysis of the release from the two different systems (NSPs/LS) were conducted. ANOVA analysis was carried out, and α=0.05 used as significant level.

The same procedures were applied for determining the loading and release profile of DOX. The DOX solution had a concentration of 2 mg/ml and the absorbance peak was at 490 nm.

Intracellular LS Release Profile of a Payload

HUVECs were seeded, grown until 70% confluence and stimulated with TNF-α at the conditions already described. HUVECs were incubated with FITC-BSA NSPs (control) and FITC-BSA LS. In these experiments the LS was realized using PE-Rhod enriched lipid membranes. After 2, 24 and 48 hr of incubation, the cells were fixed with 4% PFA solution and prepared for confocal microscopy analysis applying the staining protocol described above. The images were acquired using a Leica DM6000 upright confocal microscope equipped with a 63× oil-immersion objective.

LS Transmigration Ability

24-well transwell inserts constituted by a polycarbonate microporous membrane with a 8 μm pore size were used. HUVECs (about 2×10̂5) were seeded on the upper side of the transwell membrane and let them adhere and spread until a 100% confluence. The confluence was checked with crystal violet staining. 200 μl of media in the upper chamber was replaced with 200 μl of experimental media containing NSPs and LS (cell:particles ratio 1:5). In the lower chamber 600 μl of PBS were added in order to avoid that experimental media went through the membrane. After 24 hr, the number of loaded NSPs and LS that migrated was determined by acquiring at the microscope four nonoverlapping random fields on each well, and three wells were analyzed for each experimental point. The number of transmigrated NSPs and LS was estimated by ImageJ software.

MTT Cell Proliferation Assay

MDA-MB-231 cells were seeded in 24-well plates at 50000 cells well in EGM-2-MV media enriched with TNF-α 10 ng/ml, in a final volume of 600 μl, and transwell inserts prepared as previously described were introduced into the well. 24 hr later the media into the transwell insert was removed and substituted with 200 μl of fresh media containing NSPs and LS both preloaded with DOX, at a ratio 1:5 cell:particles. At 24, 48, 72 and 96 hr, the medium was removed and medium containing 0.5 mg/mL of 3-(4,5-dimethylthiazol-2-yl)-2,5-diphenyltetrazolium bromide (MTT, Sigma-Aldrich) was added at 200 μl well for 4 h at 37° C. to the appropriate plates. Medium was then removed and 200 μl of dimethylsulfoxide (DMSO, Sigma-Aldrich) was added to each well. After 30 min at RT, the absorbance was read at 570 nm using a SPECTRA max M2 plate reader (Molecular Devices).

### REFERENCES LIST 1

1. M. Ferrari, Nature Reviews Cancer, Vol. 5, No. 3, 2005, pp. 161-171
2. M. Ferrari, Current Opinions in Chemical Biology, Vol. 9, No. 4, August 2005, pp. 343-346
3. http://www.infoplease.com/science/health/cancer-cases-survival-rates-2006.html
4. “Cancer Facts and Figures 2008”, American Cancer Society, 2008, <http://www.cancer.org/docroot/STT/stt_{—}0_{—}2008.asp?sitearea=STT&level=1> (last accessed on Mar. 29, 2009)
5. Tasciotti E. et al, Mesoporous silicon particles as a multistage delivery system for imaging and therapeutic applications, Nature nanotechnology, 2008, 3:151-157
6. Decuzzi P et al, A theoretical model for the margination of particles within blood vessels, Ann Biomed. Eng., 2005, 33, 179-190
7. Decuzzi P et al, Fantastic Voyages, Mech. Eng., 2006, 128, 24-27
8. Decuzzi P et al, Intravascular delivery of particulate systems: does geometry really matter?, Pharm Res., 2009; 26 (1):235-43.
9. Decuzzi P et al, Adhesion of microfabricated particles on vascular endothelium: a parametric analysis, Ann Biomed. Eng., 2004, 32, 793-802
10. Huang M. Q. et al, Monitoring response to chemotherapy of non-Hodgkin's lymphoma xenografts by T2-weighted and diffusion-weighted MRI, 2008, NMR Biomed., 21:1021-1029
11. Brennan D. D. et al, A Comparison of Whole-Body MRI and CT for the Staging of Lymphoma, AJR 2005; 185:711-716
12. Cinamon G, Shinder V, Alon R. Wall shear forces promote lymphocyte migration across inflamed vascular endothelium presenting apical chemokines. Nat. Immunol. 2001; 2:515-522.
13. Yang L. et al, ICAM-1 regulates neutrophil adhesion and transcellular migration of TNF-a-activated vascular endothelium under flow, Blood, 2005, 106: 584-592.
14. Carman C et al, A transmigratory cup in leukocyte diapedesis both through individual vascular endothelial cells and between them, JCB, 2004, 167: 377-388
15. Singh H. et al, Combinib Adoptive cellular and Immunocytokine Therapies to improve treatment of B-lineage Malignacy, Cancer Res, 2007; 67: 2872-80
16. Dudley, M. E., et al., Adoptive cell therapy for patients with metastatic melanoma: evaluation of intensive myeloablative chemoradiation preparative regimens. J Clin Oncol, 2008. 26 (32): p. 5233-9.
17. Tran K Q, et al., Minimally Cultured Tumor-Infiltrating Lymphocytes Display Optimal Characteristics for Adoptive Cell Therapy. J Immunother, 2008. 31 (8): p. 742-751.
18. Powell, D. J., et al., Transition of late-stage effector T-cells to CD27+ CD28+ tumor-reactive effector memory T-cells in humans after adoptive cell transfer therapy. Blood, 2005, 105, p. 241-250
19. Singh H. et al, Redirecting Specificity of T-Cell Populations For CD19 Using the Sleeping Beauty System, Cancer Res 2008; 68: (8): 2961-2971
20. Boone C. et al, Isolation of Plasma Membrane fragments from Hela Cells, Jour Cell Biol, 1969, 41:378-392
21. Serda R. et al, The association of silicon microparticles with endothelial cells in delivery to the vasculature, Biomaterials, 2009, 30:2440-2448
22. Limseisen F M et al; differences in the physical properties of lipid monolayers and bilayers on a spherical solid support, Bioph Joum, 1997, 72, 1659-67
23. Bayerl T et al; Physical properties of single phospholipid bilayers adsorbed to micro glass beads, Biophysical Journal, 1990, 58; 357-362
24. Caracciolo G. et al, Efficient Escape from Endosomes Determines the Superior efficiency of Multicomponent Lipoplexes, 2009, IN PRESS
25. Paoloni M C and K. C, Translation of New Cancer Treatments from Pet Dogs to Humans. Nature Reviews Cancer, 2008. 8 (2): p. 147-56.

### REFERENCES LIST 2

1. Kilinc, M. O., et al., Central role of tumor associated CD8+ T effector/memory cells in restoring systemic antitumor immunity. 182, 2009. 182 (7): p. 4217-25.
2. Matsumura, S., et al., Radiation induced CXCL16 release by breast cancer cells attracts effector T-cells. J Immunol, 2008. 181 (5): p. 3099-107.
3. Pagès, F., et al., Effector memory T-cells, early metastasis, and survival in colorectal cancer. N Engl J Med, 2005. 353 (25): p. 2654-66.
4. Moghimi, S. M. and A. C. Hunter, Recognition by macrophages and liver cells of opsonized phospholipid vesicles and phospholipid headgroups. Pharm Res, 2001. 18 (1): p. 1-8.
5. Moghimi, S. M., A. C. Hunter, and J. C. Murray, Long-circulating and target-specific nanoparticles: theory to practice. Pharmacol Rev, 2001. 53 (2): p. 283-318.
6. Ferrari, M., Cancer nanotechnology: opportunities and challenges. Nat Rev Cancer, 2005. 5 (3): p. 161-171.
7. Monfardini, C. and F. M. Veronese, Stabilization of substances in circulation. Bioconjug Chem, 1998. 9 (4): p. 418-50.
8. Papisov, M. I., Theoretical considerations of RES-avoiding liposomes: Molecular mechanics and chemistry of liposome interactions. Adv Drug Deliv Rev, 1998. 32 (1-2): p. 119-138.
9. Ferrari, M., Nanovector therapeutics. Curr Opin Chem Biol, 2005. 9 (4): p. 343-6.
10. Peer, D., et al., Nanocarriers as an emerging platform for cancer therapy. Nat Nanotechnol, 2007. 2 (12): p. 751-60.
11. Sakamoto, J., et al., Antibiological barrier nanovector technology for cancer applications. Expert Opin Drug Deliv, 2007. 4 (4): p. 359-69.
12. Tanaka, T., et al., Nanotechnology for breast cancer therapy. Biomed. Microdevices, 2009. 11 (1): p. 49-63.
13. Cooper, L. J., et al., Manufacturing of gene-modified cytotoxic T lymphocytes for autologous cellular therapy for lymphoma. Cytotherapy, 2006. 8 (2): p. 105-117.
14. Singh, H., et al., Redirecting specificity of T-cell populations for CD19 using the Sleeping Beauty system. Cancer Res, 2008. 68 (8): p. 2961-71.
15. Frommolt, R., F. Rohrbach, and M. Theobald, Sleeping beauty transposon system-future trend in T-cell-based gene therapies? Future Oncol, 2006. 3 (3): p. 345-9.
16. Huang, X., et al., Stable gene transfer and expression in human primary T-cells by the Sleeping Beauty transposon system. Blood, 2006. 107 (2): p. 483-91.
17. Hackett, P. B., D. A. Largaespada, and L. J. Cooper, A Transposon and Transposase System for Human Application. Mol Ther, 2010.
18. Walter, C. U., et al., Effects of chemotherapy on immune responses in dogs with cancer. J Vet Intern Med, 2006. 20 (2): p. 342-7.
19. Rassnick, K. M., et al., Comparison of 3 protocols for treatment after induction of remission in dogs with lymphoma. J Vet Intern Med, 2007. 21 (6): p. 1364-73.
20. Chun, R., Lymphoma: which chemotherapy protocol and why?. Top Companion Anim Med, 2009. 24 (3): p. 157-62.
21. Brodsky, E. M., et al., Asparaginase and MOPP treatment of dogs with lymphoma. J Vet Intern Med, 2009. 23 (3): p. 578-84.
22. Paoloni M C and K. C, Translation of New Cancer Treatments from Pet Dogs to Humans. Nature Reviews Cancer, 2008. 8 (2): p. 147-56.
23. Slowing, II, et al., Mesoporous silica nanoparticles as controlled release delivery and gene transfection carriers. Adv Drug Deliv Rev, 2008. 60 (11): p. 1278-88.
24. Canham, L. T., Bioactive silicon structure fabrication through nanoetching techniques. Adv Mater, 1995. 7 (12): p. 1033-1037.
25. Tasciotti, E., et al., Mesoporous silicon particles as a multistage delivery system for imaging and therapeutic applications. Nat Nanotechnol, 2008. 3 (3): p. 151-7.
26. Fan, D., et al., Nano-Structured Silicon/PLGA Composite Microspheres for the Sustained Release of Biomolecules 2010.
27. Viloria-Cols, M. E., R. Hatti-Kaul, and B. Mattiasson, Agarose-coated anion exchanger prevents cell-adsorbent interactions. J Chromatogr A, 2004. 1043 (2): p. 195-200.
28. Hong, Y., et al., Collagen-coated polylactide microspheres as chondrocyte microcarriers. Biomaterials, 2005. 26 (32): p. 6305-13.
29. Royce, S. M., M. Askari, and K. G. Marra, Incorporation of polymer microspheres within fibrin scaffolds for the controlled delivery of FGF-1. J Biomater Sci Polym Ed, 2004. 15 (10): p. 1327-36.
30. Goraltchouk, A., et al., Incorporation of protein-eluting microspheres into biodegradable nerve guidance channels for controlled release. J Control Release, 2006. 110 (2): p. 400-7.
31. Freiberg, S, and X. X. Zhu, Polymer microspheres for controlled drug release. Int J Pharm, 2004. 282 (1-2): p. 1-18.
32. Yazdi, I., et al., Mesoporous silicon microparticles for the sustained delivery of antibiotics and antimicotics. In preparation, 2010.
33. Ulrich, A. S., Biophysical Aspects of Using Liposomes as Delivery Vehicles. Bioscience Reports, 2002. 22 (2): p. 129-150.
34. van de Ven, A. L., et al., Intracellular partitioning and exosomal release of nanoparticles. In preparation, 2010.
35. Tanaka, T., et al., Sustained siRNA Delivery by Mesoporous Silicon Particles for Cancer Treatment. Cancer Res, 2010. In press.
36. Serda, R. E., et al., Mitotic trafficking of silicon microparticles. Nanoscale, 2009. 1 (2): p. 250-259.
37. Tanaka, T., et al., Nanotechnology for breast cancer therapy. Biomed Microdevices, 2009. 11 (1): p. 49-63.
38. Tasciotti, E., M. Zoppe, and M. Giacca, Transcellular transfer of active HSV-1 thymidine kinase mediated by an 11-amino-acid peptide from HIV-1 Tat. Cancer Gene Ther, 2003. 10 (1): p. 64-74.
39. Tasciotti, E. and M. Giacca, Fusion of the human immunodeficiency virus type 1 tat protein transduction domain to thymidine kinase increases bystander effect and induces enhanced tumor killing in vivo. Hum Gene Ther, 2005. 16 (12): p. 1389-403.
40. Sawant, R. R. and V. P. Torchilin, Enhanced cytotoxicity of TATp-bearing paclitaxel-loaded micelles in vitro and in vivo. Int J Pharm, 2009. 374 (1-2): p. 114-8.
41. Torchilin, V. P., Cell penetrating peptide-modified pharmaceutical nanocarriers for intracellular drug and gene delivery. Biopolymers, 2008. 90 (5): p. 604-10.
42. Santra, S., et al., TAT conjugated, FITC doped silica nanoparticles for bioimaging applications. Chem Commun (Camb), 2004 (24): p. 2810-1.
43. Serrano, L. M., et al., Differentiation of naive cord-blood T-cells into CD19-specific cytolytic effectors for posttransplantation adoptive immunotherapy. Blood, 2006. 107 (7): p. 2643-52.
44. Kolonin, M. G., et al., Reversal of obesity by targeted ablation of adipose tissue. Nat Med, 2004. 10 (6): p. 625-32.
45. Ven, A. L. V. D., et al., Intracellular partitioning and exosomal release of nanoparticles. In preparation, 2010.
46. Hirsch, L. R., et al., Nanoshell-mediated near-infrared thermal therapy of tumors under magnetic resonance guidance. Proc. Natl. Acad. Sci. USA, 2003. 100 (23): p. 13549-54.
47. Gobin, A. M., et al., Near-infrared resonant nanoshells for combined optical imaging and photothermal cancer therapy. Nano. Lett., 2007. 7 (7): p. 1929-1934.
48. Michel, R. B. and M. J. Mattes, Intracellular accumulation of the anti-CD20 antibody 1F5 in B-lymphoma cells. Clin Cancer Res, 2002. 8 (8): p. 2701-13.
49. Impellizeri, J. A., et al., The role of rituximab in the treatment of canine lymphoma: an ex vivo evaluation. Vet. J, 2006. 171 (3): p. 556-8.
50. Jubala, C. M., et al., CD20 expression in normal canine B cells and in canine non-Hodgkin lymphoma. Vet Pathol, 2005. 42 (4): p. 468-76.

### REFERENCE LIST 3

1. Ratner, B. D. and S. J. Bryant, Biomaterials: where we have been and where we are going. Annu Rev Biomed Eng, 2004. 6: p. 41-75.
2. Ferrari, M., Cancer nanotechnology: opportunities and challenges. Nat Rev Cancer, 2005. 5 (3): p. 161-71.
3. Ferrari, M., Nanovector therapeutics. Curr Opin Chem Biol, 2005. 9 (4): p. 343-6.
4. Allen, T. M. and P. R. Cullis, Drug delivery systems: entering the mainstream. Science, 2004. 303 (5665): p. 1818-22.
5. Cho, K., et al., Therapeutic nanoparticles for drug delivery in cancer. Clin Cancer Res, 2008. 14 (5): p. 1310-6.
6. Moghimi, S. M., A. C. Hunter, and J. C. Murray, Long-circulating and target-specific nanoparticles: theory to practice. Pharmacol Rev, 2001. 53 (2): p. 283-318.
7. Ferrari, M., Frontiers in cancer nanomedicine: directing mass transport through biological barriers. Trends Biotechnol, 2010. 28 (4): p. 181-8.
8. Nilsson, B., et al., Can cells and biomaterials in therapeutic medicine be shielded from innate immune recognition? Trends in immunology, 2010. 31 (1): p. 32-38.
9. Peer, D., et al., Nanocarriers as an emerging platform for cancer therapy. Nat Nanotechnol, 2007. 2 (12): p. 751-60.
10. Romberg, B., W. E. Hennink, and G. Storm, Sheddable coatings for long-circulating nanoparticles. Pharm Res, 2008. 25 (1): p. 55-71.
11. Owens, D. E., 3rd and N. A. Peppas, Opsonization, biodistribution, and pharmacokinetics of polymeric nanoparticles. Int J Pharm, 2006. 307 (1): p. 93-102.
12. Ley, K., et al., Getting to the site of inflammation: the leukocyte adhesion cascade updated. Nat Rev Immunol, 2007. 7 (9): p. 678-89.
13. Nieminen, M., et al., Vimentin function in lymphocyte adhesion and transcellular migration. Nat Cell Biol, 2006. 8 (2): p. 156-62.
14. Millan, J., et al., Lymphocyte transcellular migration occurs through recruitment of endothelial ICAM-1 to caveola-and F-actin-rich domains. Nat Cell Biol, 2006. 8 (2): p. 113-23.
15. Nourshargh, S., P. L. Hordijk, and M. Sixt, Breaching multiple barriers: leukocyte motility through venular walls and the interstitium. Nat Rev Mol Cell Biol, 2010. 11 (5): p. 366-78.
16. Carman, C. V., Mechanisms for transcellular diapedesis: probing and pathfinding by ‘invadosome-like protrusions’. J Cell Sci, 2009. 122(Pt 17): p. 3025-35.
17. Yang, L., ICAM-1 regulates neutrophil adhesion and transcellular migration of TNF-a-activated vascular endothelium under flow. Blood, 2005. 106 (2): p. 584-592.
18. Azzali, G., M. L. Arcari, and G. F. Caldara, The “mode” of lymphocyte extravasation through HEV of Peyer's patches and its role in normal homing and inflammation. Microvasc Res, 2008. 75 (2): p. 227-37.
19. Tasciotti, E., et al., Mesoporous silicon particles as a multistage delivery system for imaging and therapeutic applications. Nat Nanotechnol, 2008. 3 (3): p. 151-7.
20. Decuzzi, P. and M. Ferrari, Design maps for nanoparticles targeting the diseased microvasculature. Biomaterials, 2008. 29 (3): p. 377-84.
21. Decuzzi, P., et al., Size and shape effects in the biodistribution of intravascularly injected particles. J Control Release, 2010. 141 (3): p. 320-7.
22. Chiappini, C., et al., Tailored Porous Silicon Microparticles: Fabrication and Properties. Chemphyschem, 2010.
23. Lee, S. Y., M. Ferrari, and P. Decuzzi, Design of bio-mimetic particles with enhanced vascular interaction. J Biomech, 2009. 42 (12): p. 1885-90.
24. Swanson, J. A., Shaping cups into phagosomes and macropinosomes. Nat Rev Mol Cell Biol, 2008. 9 (8): p. 639-49.
25. Novak, M. T., J. D. Bryers, and W. M. Reichert, Biomimetic strategies based on viruses and bacteria for the development of immune evasive biomaterials. Biomaterials, 2009. 30 (11): p. 1989-2005.
26. Wittchen, E. S., Endothelial signaling in paracellular and transcellular leukocyte transmigration. Front Biosci, 2009. 14: p. 2522-45.

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A composition comprising:
at least one microparticle or nanoparticle comprising at least one active agent and a surface,
wherein the surface of the at least one microparticle or nanoparticle comprises at least a portion of an isolated cellular membrane.

2. The composition of claim 1, wherein the isolated cellular membrane is an isolated plasma membrane.

3. The composition of claim 1, wherein the isolated cellular membrane is a cellular membrane isolated from a mammalian cell.

4. The composition of claim 3, wherein the isolated cellular membrane is a cellular membrane isolated from a human cell.

5. The composition of claim 1, wherein the isolated cellular membrane is a cellular membrane isolated from an immune cell.

6. The composition of claim 5, wherein the isolated cellular membrane is a cellular membrane isolated from a genetically modified immune cell.

7. The composition of claim 5, wherein the isolated cellular membrane is a cellular membrane isolated from a cell selected from the group consisting of T-cells, NK cells, monocytes and macrophages.

8. The composition of claim 1, wherein the at least one microparticle or nanoparticle comprises a lipid particle comprising a lipid layer,
wherein the lipid layer of said lipid particle comprises at least a portion of the isolated cellular membrane.

9. The composition of claim 8, wherein the at least one microparticle or nanoparticle comprises a liposome comprising a lipid layer,
wherein the lipid layer of said liposome is formed from at least a portion of the isolated cellular membrane.

10. The composition of claim 1, wherein the at least one microparticle or nanoparticle comprises a fabricated particle,
wherein at least a portion of the isolated cellular membrane is on a surface of the fabricated particle.

11. The composition of claim 1, wherein the at least one microparticle or nanoparticle comprises a porous particle,
wherein at least a portion of the isolated cellular membrane is on a surface of the porous particle.

12. The composition of claim 11, wherein the porous particle comprises at least one of porous silicon or porous silica.

13. The composition of claim 1, wherein the at least one microparticle or nanoparticle comprises a multistage object,
wherein at least a portion of the isolated cellular membrane is on a surface of the multistage object.

14. The composition of claim 1, wherein the at least one microparticle or nanoparticle is selected from the group consisting of multistage particles, porous particles, porous silicon particles, porous silica particles, non-porous particles, fabricated particles, polymeric particles, synthetic particles, semiconducting particles, viruses, gold particles, silver particles, quantum dots, indium phosphate particles, iron oxide particles, micelles, liposomes, silica particles, mesoporous silica particles, PLGA-based particles, gelatin-based particles, carbon nanotubes, fullerenes, and combinations thereof.

15. The composition of claim 1, wherein the at least one microparticle or nanoparticle comprises a particle with a functionalized surface.

16. The composition of claim 15, wherein the surface of the particle is functionalized with a functionalizing agent selected from the group consisting of peptides, polymers, chitosans, contrasting agents, imaging agents and calcium phosphates.

17. The composition of claim 15, wherein the surface of the particle is functionalized with a polymer, wherein the polymer becomes swellable in response to a stimulus selected from the group consisting of change in temperature, change in pH, change in pressure, and combinations thereof.

18. The composition of claim 1, wherein the active agent comprises a therapeutic agent.

19. The composition of claim 18, wherein the therapeutic agent is selected from the group consisting of anti-inflammatory agents, anti-cancer agents, anti-proliferative agents, anti-vascularization agents, wound repair agents, tissue repair agents, thermal therapy agents, and combinations thereof.

20. The composition of claim 1, wherein the active agent comprises an imaging agent.

21. The composition of claim 1, wherein the active agent is on the surface of the at least one microparticle or nanoparticle.

22. The composition of claim 1, wherein the active agent is inside the at least one microparticle or nanoparticle.

23. The composition of claim 1, wherein the composition is used to treat, monitor, diagnose, or prevent a condition associated with inflammation.

24. The composition of claim 23, wherein the condition to be treated, monitored, diagnosed, or prevented is cancer.

25. A method of making a delivery system comprising:
(a) isolating a cellular membrane from a cell; and
(b) associating at least a portion of the isolated cellular membrane with a surface of a microparticle or a nanoparticle, thereby forming the delivery system.

26. The method of claim 25, wherein said associating comprises disposing at least a portion of the isolated cellular membrane on a surface of the microparticle or nanoparticle.

27. The method of claim 25, wherein the isolating comprises isolating and purifying at least a portion of the cellular membrane by ultracentrifugation through a discontinuous sucrose density gradient.

28. The method of claim 25, wherein the isolated cellular membrane is a plasma membrane.

29. The method of claim 25, wherein the cell is a mammalian cell.

30. The method of claim 25, wherein the cell is a human cell.

31. The method of claim 25, wherein the cell is an immune cell.

32. The method of claim 31, wherein the isolated cellular membrane is a cellular membrane isolated from a genetically modified immune cell.

33. The method of claim 31, wherein the cell is selected from the group consisting of T-cells, NK cells, monocytes and macrophages.

34. The method of claim 25, wherein said forming comprises forming a lipid particle,
wherein a lipid layer of said lipid particle comprises at least a portion of the isolated cellular membrane.

35. The method of claim 34, wherein the lipid particle is a liposome.

36. The method of claim 25, further comprising obtaining a microparticle or a nanoparticle.

37. The method of claim 36, wherein said obtaining comprises fabricating said microparticle or nanoparticle.

38. The method of claim 25, wherein said microparticle or nanoparticle is a porous particle.

39. The method of claim 38, wherein said porous particle is at least one of a porous silicon particle or a porous silica particle.

40. The method of claim 39, further comprising loading at least one active agent in pores of the porous particle prior to the associating of the isolated cellular membrane.

41. The method of claim 36, further comprising disposing an adhesive agent on the surface of the obtained microparticle or nanoparticle prior to the associating of at least a portion of the isolated membrane.

42. The method of claim 36, wherein said obtaining comprises obtaining a multistage object comprising a first stage particle containing at least one second stage particle.

43. The method of claim 42, wherein said obtaining the multistage object comprises obtaining the first stage particle and loading the at least one second stage particle into the first stage particle.

44. The method of claim 43, wherein said disposing comprises incubation of the obtained particle in a medium comprising at least a portion of the isolated membrane.

45. The method of claim 25, wherein the at least one microparticle or nanoparticle is selected from the group consisting of multistage particles, porous particles, porous silicon particles, porous silica particles, non-porous particles, fabricated particles, polymeric particles, synthetic particles, semiconducting particles, viruses, gold particles, silver particles, quantum dots, indium phosphate particles, iron oxide particles, micelles, liposomes, silica particles, mesoporous silica particles, PLGA-based particles, gelatin-based particles, carbon nanotubes, fullerenes, and combinations thereof.

46. The method of claim 25, wherein the active agent comprises a therapeutic agent.

47. The method of claim 46, wherein the therapeutic agent is selected from the group consisting of anti-inflammatory agents, anti-cancer agents, anti-proliferative agents, anti-vascularization agents, wound repair agents, tissue repair agents, thermal therapy agents, and combinations thereof.

48. The method of claim 25, wherein the active agent comprises an imaging agent.

49. The method of claim 25, wherein the active agent is on the surface of the at least one microparticle or nanoparticle.

50. The method of claim 25, wherein the active agent is inside the at least one microparticle or nanoparticle.

51. The method of claim 25, wherein the delivery system is used to treat, monitor, diagnose, or prevent a condition associated with inflammation.

52. The method of claim 51, wherein the condition to be treated, monitored, diagnosed, or prevented is cancer.

53. A delivery method comprising:
administering to a subject a composition comprising:
at least one microparticle or nanoparticle comprising at least one active agent and a surface,
wherein the surface of the at least one microparticle or nanoparticle comprises at least a portion of an isolated cellular membrane.

54. The delivery method of claim 53, wherein the isolated cellular membrane is a plasma membrane.

55. The delivery method of claim 53, wherein the cellular membrane is derived from a mammalian cell.

56. The delivery method of claim 55, wherein the cell is a human cell.

57. The delivery method of claim 56, wherein the cell is an immune cell.

58. The delivery method of claim 57, wherein at least a portion of the isolated cellular membrane is a cellular membrane isolated from a genetically modified immune cell.

59. The delivery method of claim 57, wherein the cell is selected from the group consisting of T-cells, NK cells, monocytes and macrophages.

60. The delivery method of claim 53, wherein the administering comprises at least one of intravenous administration, subcutaneous administration, and intramuscular administration.

61. The delivery method of claim 53, wherein the subject is a human being suffering from a condition associated with inflammation.

62. The delivery method of claim 61, wherein the condition associated with inflammation is cancer.

63. The delivery method of claim 61, wherein the composition migrates to a site associated with the condition within the subject after administration.

64. The delivery method of claim 63, wherein the active agent is released from the composition after migration to the site associated with the condition.

65. The delivery method of claim 53, wherein said at least one microparticle or nanoparticle comprises a lipid particle,
wherein a lipid layer of said lipid particle comprises at least a portion of the isolated cellular membrane.

66. The method of claim 53, wherein the at least one microparticle or nanoparticle comprises a liposome,
wherein a lipid layer of said liposome is formed from at least a portion of the isolated cellular membrane.

67. The delivery method of claim 53, wherein the at least one microparticle or nanoparticle comprises a fabricated particle,
wherein at least a portion of the isolated cellular membrane is on a surface of the fabricated particle.

68. The delivery method of claim 53, wherein the at least one microparticle or nanoparticle comprises a porous particle,
wherein at least a portion of the isolated cellular membrane is on a surface of the porous particle.

69. The delivery method of claim 53, wherein the at least one microparticle or nanoparticle comprises a multistage object,
wherein at least a portion of the isolated cellular membrane is on a surface of the multistage object.

70. The delivery method of claim 53, wherein the active agent comprises a therapeutic agent.

71. The delivery method of claim 70, wherein the therapeutic agent is selected from the group consisting of anti-inflammatory agents, anti-cancer agents, anti-proliferative agents, anti-vascularization agents, wound repair agents, tissue repair agents, thermal therapy agents, and combinations thereof.

72. The delivery method of claim 53, wherein the active agent comprises an imaging agent.
